(19) United States
(12) Patent Application Publication
(10) Pub. No.: US 2020/0046562 A1
(43) Pub. Date: Feb. 13, 2020

(54) FINGERTIP CLEANING APPARATUS

(71) Applicant: FINGERTIPS ENTERPRISES, LLC, New York, NY (US)

(72) Inventors: Alon Seifert, New York, NY (US); Eitan Cohen, New York, NY (US); Matthew L. Chin, New York, NY (US); Gareth Brown, Jersey City, NJ (US); Stephen Kaes, New York, NY (US)

(21) Appl. No.: 16/598,731

(22) Filed: Oct. 10, 2019

Related U.S. Application Data
(63) Continuation-in-part of application No. 15/494,084, filed on Apr. 21, 2017, now Pat. No. 10,449,093.
(60) Provisional application No. 62/743,865, filed on Oct. 10, 2018.
(60) Provisional application No. 62/325,600, filed on Apr. 21, 2016.

(51) Int. Cl. A61F 11/00

(52) U.S. Cl. CPC A61F 11/006; A61B 2017/00438

(57) ABSTRACT

A fingertip cleaning apparatus having a three-dimensional structure. The three-dimensional structure includes a fingertip receiving portion having a conical wall, a rounded apex wall at a distal end of the conical wall, a cavity having a top end that is closed by the rounded apex wall, an opening into the cavity defined, at least in part, by a lower edge of the conical wall, and a gripping portion including a flap wall that extends from a proximal end of the conical wall of the fingertip receiving portion.

### CROSS-REFERENCE TO RELATED APPLICATIONS

This application claims the benefit of U.S. Provisional Patent Application Ser. No. 62/743,865, filed on Oct. 10, 2018, and is a continuation-in-part of U.S. patent application Ser. No. 15/494,084, filed on Apr. 21, 2017, which claims the benefit of U.S. Provisional Patent Application Ser. No. 62/325,600, filed on Apr. 21, 2016, the entireties of which are incorporated herein by reference.

### FIELD OF THE INVENTION

The present invention generally relates to cleaning devices placed over the finger for cleaning body surfaces and/or cavities.

### BACKGROUND OF THE INVENTION

Earwax, also known by the medical term cerumen, is a yellowish waxy substance secreted in the ear canal of humans and other mammals. It protects the skin of the human ear canal, assists in cleaning and lubrication, and also provides some protection from bacteria, fungi, insects, and water.

Earwax is good for the overall health of the ear, a fact to which practically every doctor will attest. However, nobody seems to care about what doctors think when it comes to earwax and earwax build-up. Almost everybody cleans their ears for aesthetic reasons, even at the price of going against their doctor's advice, and most people have absolutely no desire to change this personal grooming preference. There are generally no products on the market that are designed specifically to clean earwax from the ear canal. As a result, people will stick almost anything in their ears to get the wax out. The tool of choice for most people is the common cotton swab, which turns out to be a very dangerous tool for inserting into the ear canal to clean out earwax. Cotton swabs can push earwax deeper into the ear canal, thereby causing further wax buildup, pain, itching, a risk of infection, and hearing loss. Another reason why a person shouldn't remove the wax with a cotton swab in particular is that at the end of the ear canal is the eardrum, and the eardrum is much closer to the outside than most people think. A cotton swab pushed too far into the ear canal can easily damage the eardrum, sometimes resulting in permanent hearing loss. For these reasons, a more useful and appealing alternative to a cotton swab is needed.

### SUMMARY OF THE INVENTION

The present invention is directed toward a fingertip cleaning apparatus comprising a fingertip receiving portion including a conical wall defining a cavity having an open end configured to allow a fingertip of a user to be inserted into the cavity, the conical wall extending along and surrounding a cone axis, the conical wall forming a cone angle with the cone axis; a gripping portion comprising a flap that extends from the conical wall of the fingertip receiving portion along a grip axis that forms a grip angle with the cone axis; and the grip angle being greater than the cone angle. The fingertip cleaning apparatus may be designed to fit over the tip of a user's finger and then inserted into the hear for cleaning. The fingertip cleaning apparatus may also be used to clean exterior parts of the ear, such as the Scapha, Anti Helix, Concha, Anti Tragus, Lobule, Superior and Inferior Crus, and Helix thereof

In one embodiment, the invention may be a fingertip cleaning apparatus comprising: a three-dimensional structure comprising: a fingertip receiving portion comprising a conical wall, a rounded apex wall at a distal end of the conical wall, a cavity having a top end that is closed by the rounded apex wall, and an opening into the cavity defined, at least in part, by a lower edge of the conical wall; and a gripping portion comprising a flap wall that extends from a proximal end of the conical wall of the fingertip receiving portion; wherein the three-dimensional structure is formed from a single sheet of fabric press molded into a shape-retaining structure.

In another embodiment, the invention may be a fingertip cleaning apparatus comprising: a three-dimensional structure comprising: a fingertip receiving portion comprising a conical wall, an apex wall at a distal end of the conical wall, a cavity having a top end that is closed by the apex wall, and an opening into the cavity defined, at least in part, by a lower edge of the conical wall; and a gripping portion comprising a flap wall that extends from a proximal end of the conical wall of the fingertip receiving portion; and wherein the three-dimensional structure is free of seams.

In yet another embodiment, the invention may be a fingertip cleaning apparatus comprising: a three-dimensional structure comprising: a fingertip receiving portion comprising a conical wall, an apex wall at a distal end of the conical wall, a cavity having a top end that is closed by the apex wall, and an opening into the cavity defined, at least in part, by a lower edge of the conical wall; and a gripping portion comprising a flap wall that extends from a proximal end of the conical wall of the fingertip receiving portion, the flap wall comprising a first wall thickness measured from an exposed inner surface of the flap wall to an exposed outer surface of the flap wall; and wherein at least one of the apex wall and the conical wall comprise a second wall thickness measured from an exposed inner surface of the fingertip receiving portion to an exposed outer surface of the fingertip receiving portion, the second wall thickness being less than the first wall thickness.

In a further embodiment, the invention may be a fingertip cleaning apparatus comprising: a three-dimensional structure comprising: a fingertip receiving portion comprising a conical wall, an apex wall at a distal end of the conical wall, a cavity having a top end that is closed by the apex wall, and an opening into the cavity defined, at least in part, by a lower edge of the conical wall; and a gripping portion comprising a flap wall that extends from a proximal end of the conical wall of the fingertip receiving portion, wherein the flap wall has a first stiffness and the conical wall has a second stiffness that is greater than the first stiffness.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The foregoing summary, as well as the following detailed description of the exemplary embodiments, will be better understood when read in conjunction with the appended drawings. It should be understood, however, that the invention is not limited to the precise arrangements and instrumentalities shown in the following figures:

FIG. 1 is a front perspective view of a fingertip cleaning apparatus in accordance with an embodiment of the present invention;

FIG. 2 is a rear perspective view of the fingertip cleaning apparatus of FIG. 1;

FIG. 3 is a rear elevation view of the fingertip cleaning apparatus of FIG. 1;

FIG. 4 is a side elevation view of the fingertip cleaning apparatus of FIG. 1;

FIG. 5 is a front elevation view of the fingertip cleaning apparatus of FIG. 1;

FIGS. 6A-6B are cross-sectional views taken along line IX-IX of FIG. 3;

FIGS. 7A-7C are rear, front, and side views of a fingertip cleaning apparatus according to another embodiment of the present invention;

FIGS. 8A-8D are cross-sectional views taken along line IX-IX of FIG. 3;

FIG. 9A is rear elevation view of a fingertip cleaning apparatus according to another embodiment of the present invention;

FIG. 9B is cross-sectional view taken along line X-X of FIG. 9A;

FIGS. 10A and 10B are different embodiments of the close-up view of the section X depicted in FIG. 9B;

FIGS. 11A and 11B illustrates different grips which may be used with the fingertip cleaning apparatus of the present invention;

FIG. 12 is the fingertip cleaning apparatus of the present invention being used to clean the ear canal;

FIG. 13 illustrates the fingertip cleaning apparatus of the present invention being used to clean the auricle;

FIG. 14A is a top view of a sheet blank according to one embodiment of the present invention;

FIG. 14B is a front view of a fingertip cleaning apparatus formed from the sheet blank of FIG. 14A;

FIG. 15A is a top view of a sheet blank according to another embodiment of the present invention;

FIG. 15B is a rear perspective view of a fingertip cleaning apparatus formed from the sheet blank of FIG. 15A;

FIGS. 16-18 illustrate a method of forming the fingertip cleaning apparatus in accordance with an embodiment of the present invention;

FIG. 19 illustrates a method of treating the fingertip cleaning apparatus of the present invention;

FIG. 20 illustrates a plurality of the fingertip cleaning apparatus of the present invention in a stacked arrangement;

FIGS. 21A and 22B illustrate front and sectional views of the fingertip cleaning apparatus according to another embodiment of the present invention;

FIG. 22 is a kit comprising a plurality of the fingertip cleaning apparatus of the present invention;

FIG. 23 is a side view of the fingertip cleaning apparatus of the present invention;

FIG. 24 is a rear view of the fingertip cleaning apparatus according to another embodiment of the present invention;

FIG. 25A is an exploded view of the fingertip cleaning apparatus according to another embodiment of the present invention;

FIG. 25B an assembled view of the fingertip cleaning apparatus of FIG. 25A;

FIG. 26 is an exploded view of the fingertip cleaning apparatus according to another embodiment of the present invention;

FIG. 27 is an exploded view of the fingertip cleaning apparatus according to another embodiment of the present invention;

FIG. 28 is an exploded view of the fingertip cleaning apparatus according to another embodiment of the present invention;

FIG. 29 is an exploded view of the fingertip cleaning apparatus according to another embodiment of the present invention;

FIG. 30 is an exploded view of the fingertip cleaning apparatus according to another embodiment of the present invention;

FIG. 31 is an exploded view of the fingertip cleaning apparatus according to another embodiment of the present invention;

FIG. 32 is an exploded view of the fingertip cleaning apparatus according to another embodiment of the present invention;

FIG. 33 is an elevated front perspective view of a fingertip cleaning apparatus according to yet another embodiment of the invention;

FIG. 34 is a depressed rear perspective view of the fingertip cleaning apparatus of FIG. 33;

FIG. 35 is a left-side plan view of the fingertip cleaning apparatus of FIG. 33, the right-side plan view being a mirror image thereof;

FIG. 36 is a bottom plan view of the fingertip cleaning apparatus of FIG. 33;

FIG. 37 is a front plan view of the fingertip cleaning apparatus of FIG. 33;

FIG. 38 is a cross-sectional view taken along line XXXVIII-XXXVIII of FIG. 37;

FIG. 39 is a cross-sectional view taken along line XXXIX-XXXIX of FIG. 35;

FIG. 40 is a cross-sectional view taken along line XXXX-XXXX of FIG. 35;

FIG. 41 is a schematic of a manufacturing lines/process for producing the fingertip cleaning apparatus of FIG. 33 according to an embodiment of the invention;

FIGS. 42A-C illustrate a panel cutting station of the manufacturing line/process of FIG. 41 cutting panels from a roll of fabric;

FIG. 43 is a schematic of a press of the manufacturing line/process of FIG. 41 with the panels formed in FIGS. 42A-42C positioned above a female portion of a mold of the press;

FIG. 44A-C illustrate the press of FIG. 43 forming the panels into the shape-retaining three-dimensional structure of the fingertip cleaning apparatus of FIG. 33;

FIG. 45 is a schematic of a final shape cutting station of the manufacturing line/process of FIG. 41 with the shape-retaining three-dimensional structures formed in the press positioned therein;

FIGS. 46A-C illustrate the final shape cutting station of FIG. 45 cutting the three-dimensional structures into their final shape;

FIG. 47 is a schematic of a stacking station of the manufacturing line/process of FIG. 41 with the finally-shaped three-dimensional structures positioned therein;

FIGS. 48A-B illustrate the stacking station of FIG. 47 stacking the finally-shaped three-dimensional structures in stacked and nesting arrangements within a tray;

FIGS. 49A-B illustrate the stacked and nesting arrangements of the fingertip cleaning apparatus being wetted in a wetting station of the manufacturing line/process of FIG. 41;

FIGS. 50A-B illustrate the wetted stacked and nesting arrangements of the fingertip cleaning apparatus being packaged in a packaging station of the manufacturing line/process of FIG. 41; and

FIG. 51 is a cross-sectional schematic of a final packaged product of the fingertip cleaning apparatus in accordance with an embodiment of the present invention.

### DETAILED DESCRIPTION OF THE INVENTION

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “left,” “right,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the preferred embodiments. Accordingly, the invention expressly should not be limited to such preferred embodiments illustrating some possible non-limiting combinations of features that may exist alone or in other combinations of features; the scope of the invention being defined by the claims appended hereto.

According to the present invention the term “about” refers to a value that is ±3% of the referenced value. According to the present invention the phrase “substantially equal” refers to a value that has a difference in value less than or equal to 3% of the referenced value. According to the present invention, the phrase “substantially parallel” refers to an angle of 0°±2°. The phrases “substantially perpendicular” and “substantially orthogonal” refers to an angle of 90°±2°.

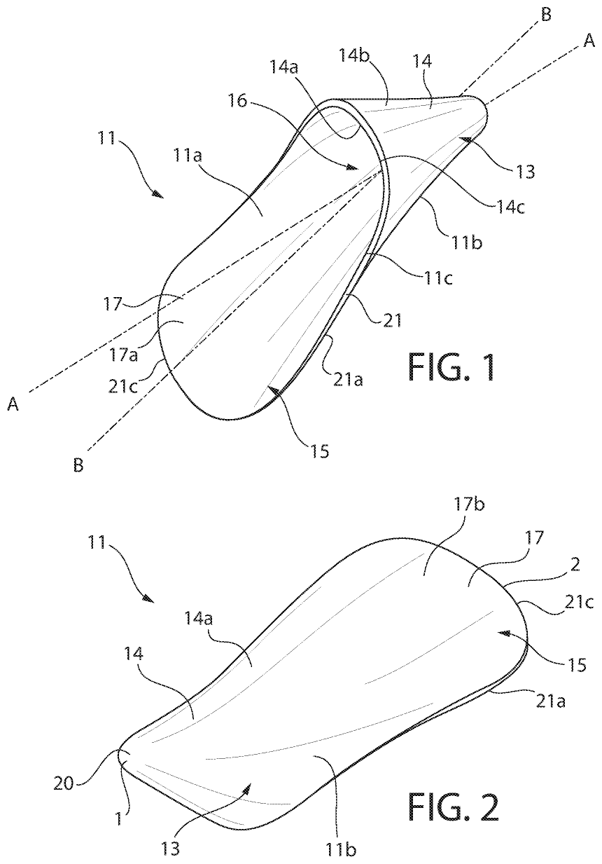

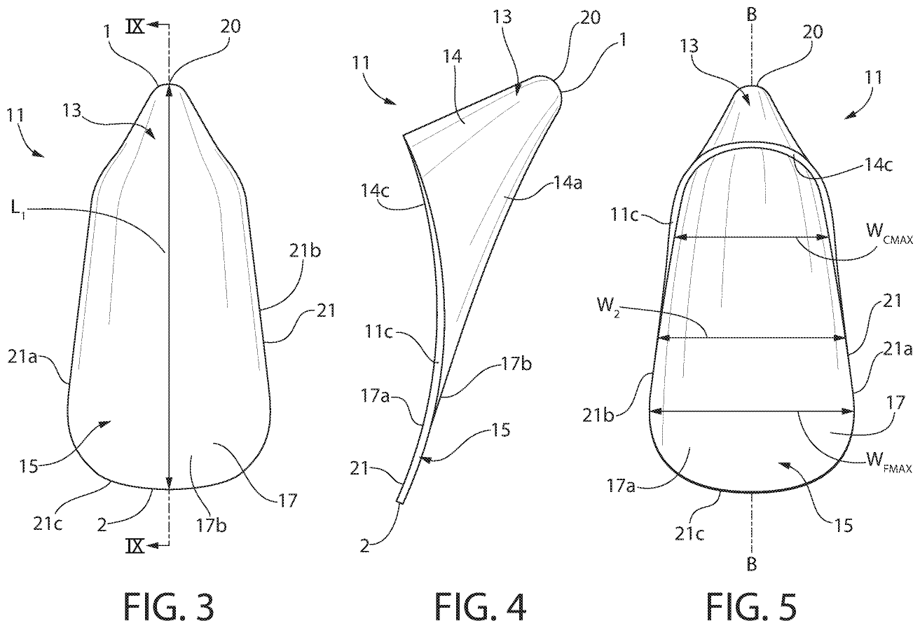

Turning in detail to the drawings, FIGS. 1-6 illustrates a fingertip cleaning apparatus 11 according to the present invention. The fingertip cleaning apparatus 11 is configured to be placed over a user's index finger for cleaning earwax, dirt, and other unwanted contaminants from various external body surfaces. Non-limiting examples of suitable external body surfaces include ear, nose, forehead, neck, armpit, and toes. Although not limited to ear cleaning, the fingertip cleaning apparatus 11 of the present invention will be referred to herein as an “ear cleaning apparatus” 11, which is configured to be placed over a user's index finger for cleaning earwax, dirt, and other unwanted contaminants from the outer part of the ear canal—as described further herein.

The ear cleaning apparatus 11 includes a fingertip receiving portion 13 (also referred to as an “ear insertion portion” or “insertion portion”) and a gripping portion 15. The ear cleaning apparatus 11 may comprise a flexible sheet 80, 80a, 80b that is folded into a three-dimensional configuration that forms the fingertip receiving portion 13 and the gripping portion 15—as discussed further herein. In some embodiments, the fingertip receiving portion 13 and the gripping portion 15 are formed from a single flexible sheet 80, 80a, 80b that is folded into the ear cleaning apparatus 11—as discussed further herein.

The ear cleaning apparatus 11 may comprise an inner surface 11a, an outer surface 11b, and an edge 11c—the edge 11 extending between the inner surface 11a and the outer surface 11b. The ear cleaning apparatus 11 may comprise a distal end 1 opposite a proximal end 2. The ear cleaning apparatus 11 may have a length L₁ that is measured from the distal end 1 to the proximal end 2. Non-limiting examples of the length L₁ may range from about 35 mm to about 85 mm—including all lengths and sub-ranges there-between. In some embodiments, the length L₁ may range from about 55 mm to about 70 mm—including all lengths and sub-ranges there-between.

The fingertip receiving portion 13 includes a conical wall 14 defining a cavity 16 having an open end 18 configured to allow a fingertip of a user to be inserted into the cavity 16. The conical wall 14 may comprise an inner surface 14a opposite an outer surface 14b. The conical wall 14 may further comprise a lower edge 14c that extends between the inner surface 14a and the outer surface 14b of the conical wall 14. The conical wall 14 extends along and surrounding a cone axis A-A. The cavity 16 further comprises a closed end 19 that is opposite the open end 18 along the cone axis A-A.

The conical wall 14 forms a cone angle Ø₁ with the cone axis A-A. The cone angle Ø₁ may range from about 15° to about 65°—including all angles and sub-ranges there-between. In a preferred embodiments, the cone angle Ø₁ may range from about 25° to about 50°—including all angles and sub-ranges there-between. In a preferred embodiments, the cone angle Ø₁ may range from about 35° to about 45°—including all angles and sub-ranges there-between.

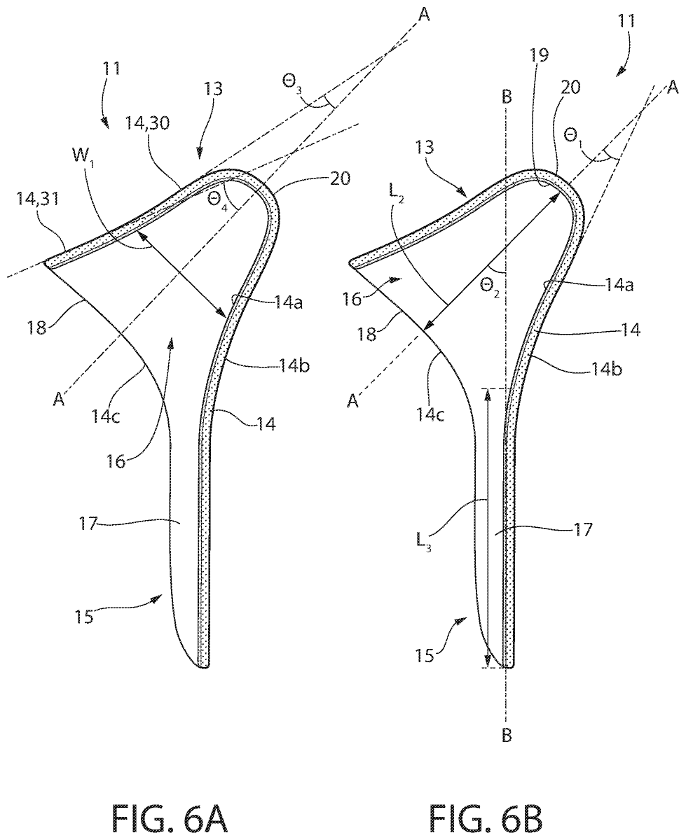

The open end 18 of the cavity 16 may be at least partially circumscribed by the lower edge 14c of the conical wall 14. The lower edge 14c may partially define the open end 18 of the cavity 16. As demonstrated by FIG. 6, at least a portion of the lower edge 14c may be oriented substantially perpendicular to the cone axis A-A.

The conical wall 14 may comprise an apex 20. The distal end 1 of the ear cleaning apparatus 11 may comprise the apex 20 of the conical wall 14. The apex 20 may be rounded (also referred to as a “rounded apex”) such that distal end 1 of the ear cleaning apparatus 11 does not comprise a point. The closed end 19 of the cavity 16 may be defined by the apex 20 of the conical wall 14. The closed end 19 may form a ceiling of the cavity 16, whereby the ceiling also comprises a rounded geometry.

Referring now to FIG. 6A, in some embodiments the conical wall 14 may comprise an upper portion 30 and a lower portion 31. The upper portion 30 of the conical wall 14 may comprise the apex 20. The lower portion 31 of the conical wall 14 may at least partially define the open end 18 of the cavity 16. In some embodiments, the upper portion may comprise the apex 20 and closed end 19 and extend to the lower portion 31, whereby the lower portion 31 comprises the open end 18. The upper portion may form a first cone angle Ø₃ with the cone axis A-A. The lower portion may form a second cone angle Ø₄ with the cone axis A-A.

The second cone angle Ø₄ of the lower portion 31 of the conical wall 14 may be equal to or greater than the first cone angle Ø₃ of the upper portion 30 of the conical wall 14. In some embodiments, the second cone angle Ø₄ of the lower portion 31 of the conical wall 14 may be greater than the first cone angle Ø₃ of the upper portion 30 of the conical wall 14. A ratio between the second cone angle Ø₄ and the first cone angle Ø₃ may range from about 1.0:1.0 to about 5.0:1.0—including all ratios and sub-ranges there-between. In other embodiments, the ratio between the second cone angle Ø₄ and the first cone angle Ø₃ may range from about 1.1:1.0 to about 4.0:1.0—including all ratios and sub-ranges there-between.

Referring to FIG. 6B, the conical wall 14 may have a length L₂ as measured along the cone axis A-A from the open end 18 to the apex 20 (or closed end 19) of the cavity 16. The length L₂ of the conical wall 14 may range from about 15 mm to about 35 mm—including all lengths and sub-ranges there-between. In a non-limiting example, the length L₂ of the conical wall 14 may be about 24 mm.

As demonstrated by FIG. 6A, the cavity 16 may have a width W₁ that is the distance between opposite inner surfaces 14a of the conical wall 14 as measured in a direction that is substantially perpendicular to the cone axis A-A. The width W₁ of the cavity 16 may range from about 5 mm to about 30 mm—including all widths and sub-ranges there-between. In some embodiments, the width W₁ of the cavity 16 may range from about 7 mm to about 20 mm—including all widths and sub-ranges there-between.

The width W₁ of the cavity 16 may be non-constant when viewed along the cone axis A-A. The width W1 of the cavity 16 may taper moving in a direction from the open end 18 to the closed end 19 of the cavity 16. The width W₁ of the cavity 16 may taper in a linear fashion. In other embodiments, the width W₁ of the cavity 16 may taper in a non-linear fashion. The width W₁ of the cavity 16 proximate to the open end 18 may be greater than the width W₁ of the cavity 16 at the closed end 19. The width W₁ proximate the open end 18 of the cavity 16 may be about 20 mm. The width W₁ proximate the closed end 19 of the cavity 16 may be about 7 mm.

The conical wall 14 may have a cross-sectional shape that is taken along the cone axis A-A which is circular. In other embodiments, the conical wall 14 may have a cross-sectional shape that is taken along the cone axis A-A which is ovular. In other embodiments, the conical wall 14 may have a cross-sectional shape taken along the cone axis A-A that is polygonal—including but not limited to 3, 4, 5, 6, 7, 8, 9, or 10 distinct sides. The polygonal cross-sectional shape may be a regular polygon (also referred to as an equiangular polygon) or an irregular polygon.

The gripping portion 15 comprises a flap 17. The flap 17 may extend directly from the fingertip receiving portion 13, and it may be formed as an extension of the fingertip receiving portion 13 to have a flat to slightly curved shape. The flap 17 extends from the conical wall 14 of the fingertip receiving portion 13 along a grip axis B-B. The grip axis B-B forms a grip angle Ø₂ with the cone axis A-A. The grip angle Ø₂ may range from about 15° to about 80°—including all angles and sub-ranges there-between. In a preferred embodiments, the grip angle Ø₂ may range from about 25° to about 75°—including all angles and sub-ranges there-between. In a preferred embodiments, the grip angle Ø₂ may range from about 40° to about 70°—including all angles and sub-ranges there-between.

The grip angle Ø₂ may be greater than or equal to the cone angle Ø₁. In some embodiments, the grip angle Ø₂ may be substantially equal to the cone angle Ø₁—as discussed further herein. In other embodiments, the grip angle Ø₂ may be greater than the cone angle Ø₁—as discussed further herein. A ratio between the grip angle Ø₂ and the cone angle Ø₁ may range from about 1.0:1.0 to about 8.0:1.0—including all ratios and sub-ranges there-between. In other embodiments, the ratio between the grip angle Ø₂ and the cone angle Ø₁ may range from about 1.1:1.0 to about 4.0:1.0—including all ratios and sub-ranges there-between.

The flap 17 may comprise curved transverse cross-sectional profile taken along the grip axis B-B. The flap 17 may comprise an inner surface 17a opposite an outer surface 17b, as well as a side edge 21 extending there-between. The inner surface 17a of the flap 17 may be concave in shape. The outer surface 17b of the flap 17 may be convex in shape.

The side edge 21 of the flap 17 may comprise a first side edge 21a, a second side edge 21b, and a lower edge 21c. The lower edge 21c may extend between the first side edge 21a and the second side edge 21b. The first side edge 21a may extend continuously into the lower edge 21c. The second side edge 21b may extend continuously into the lower edge 21c. The first edge 21a, second side edge 21b, and the lower edge 21c may collectively define an uninterrupted surface that forms the side edge 21 of the flap 17.

Referring to FIG. 6B, the flap 17 may have a length L₃ as measured along the grip axis B-B from the open end 18 of the cavity 16 to the distal-most end of the flap (show as the proximal end 2 of the of the ear cleaning apparatus 11 in FIG. 6b). The length L₃ of the flap 17 may range from about 30 mm to about 50 mm—including all lengths and sub-ranges there-between. In a non-limiting example, the length L₃ of the flap 17 may be about 36 mm.

The length L₃ of the flap 17 may be equal to or greater than the length L₂ of the conical wall 14. In some embodiments, the length L₃ of the flap 17 may be greater than the length L₂ of the conical wall 14. A ratio of the length L₃ of the flap 17 to the length L₂ of the conical wall 14 may range from about 1.0:1.0 to about 3.0:1.0—including all ratios and sub-ranges there-between. In a preferred embodiment, the ratio of the length L₃ of the flap 17 to the length L₂ of the conical wall 14 may range from about 1.1:1.0 to about 2.5:1.0—including all ratios and sub-ranges there-between.

As demonstrated by FIG. 5, the flap 18 may have a width W₂ that is the distance between the first side edge 21a and the second side edge 21b as measured in a direction that is substantially perpendicular to the grip axis B-B. The width W₂ of the flap 18 may range from about 20 mm to about 50 mm—including all widths and sub-ranges there-between. In some embodiments, the width W₂ of the flap 18 may range from about 20 mm to about 40 mm—including all widths and sub-ranges there-between.

The width W₁ of the flap 18 may be non-constant when viewed along the grip axis B-B. The width W1 of the flap 16 may taper moving in a direction from the distal end 1 to the proximal end 2 of ear cleaning apparatus 11. The width W₂ of the flap 18 may taper in a linear fashion. In other embodiments, the width W₂ of the flap 18 may taper in a non-linear fashion. The width W₂ of the flap 18 adjacent the proximal end 2 may be greater than the width W₂ of the flap 18 adjacent the insertion portion 13. The width W₂ of the flap 18 adjacent the proximal end 2 may be about 30 mm. The width W₂ of the flap 18 adjacent the insertion portion 13 may be about 24 mm.

The flap 18 may have a first maximum width W_{FMAX} and the cavity 16 may have a second maximum width W_{CMAX}. The first maximum width W_{FMAX} of the flap 18 may be equal to or greater than the second maximum width W_{CMAX} of the cavity 16 In a preferred embodiment, the first maximum width W_{FMAX} of the flap 18 is greater than the second maximum width W_{CMAX} of the cavity 16. A ratio of the first maximum width W_{FMAX} of the flap 18 and the second maximum width W_{CMAX} of the cavity 16 may range from about 1:1 to about 3:1—including all ratios and sub-ranges there-between. In a preferred embodiment, the ratio of the first maximum width W_{FMAX} of the flap 18 to the second maximum width W_{CMAX} of the cavity 16 may range from about 1.1:1 to about 2:1—including all ratios and sub-ranges there-between.

The flap 17 may extend from the conical wall 14 such that the outer surface 17b of the flap 17 is continuous with the outer surface 14b of the conical wall 14 and collectively defining an uninterrupted surface. In some embodiments, the flap 17 may extend from the conical wall 14 such that the outer surface 17b of the flap 17 is continuous with the outer surface 14b of the conical wall 14 and collectively defining an uninterrupted surface that forms the outer surface 11b of the ear cleaning apparatus 11. The uninterrupted outer surface 11b of the ear cleaning apparatus 11 may be smooth.

The side surface 11c of the ear cleaning apparatus 11 may comprise the lower edge 14c of the conical wall 14. The side surface 11c of the ear cleaning apparatus 11 may comprise the side edge 21 of the flap 17. The lower edge 14c of the conical wall 14 may extend into the side edge 21 of the flap 17. In some embodiments, the lower edge 14c of the conical wall 14 may extend continuously into the side edge 21 of the flap 17 to form the side surface 11c of the ear cleaning apparatus 11, whereby the side surface 11c of is continuous and uninterrupted. The first side edge 21a, second side edge 21b, and lower edge 21c of the flap 17 as well as the lower edge 14c of the conical wall 14 may define a continuous edge 11c of the ear cleaning apparatus 11.

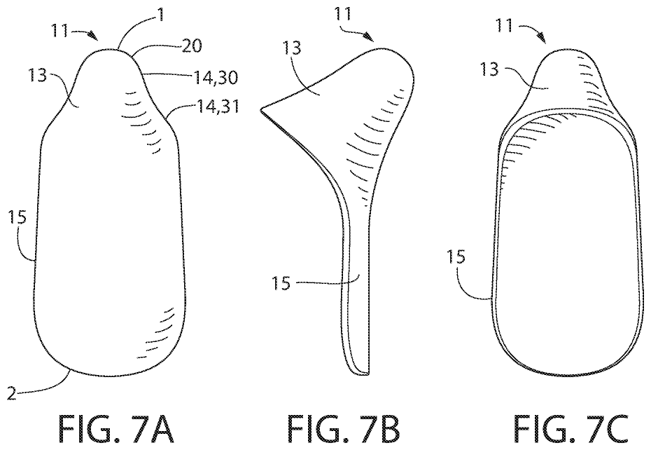

Referring now to FIGS. 7A-7C, another embodiment of the ear cleaning apparatus 11 is provided. As compared to the embodiment shown in FIGS. 1-6, the fingertip receiving portion 13 is formed to have a larger cross-sectional circumference when viewed in a plane which is orthogonal to the cone axis A-A of the fingertip receiving portion 13. The tip of the fingertip receiving portion 13 of this ear cleaning apparatus 11 is also broader than the tip of the fingertip receiving portion 13 of the ear cleaning apparatus 11 of FIGS. 1-6. This ear cleaning apparatus 11 may therefore be made for a user having larger fingers, or alternatively, it may be intended for use by all users, although it would not extend as far into the ear canal during use as would the fingertip receiving portion 13 of the ear cleaning apparatus 11.

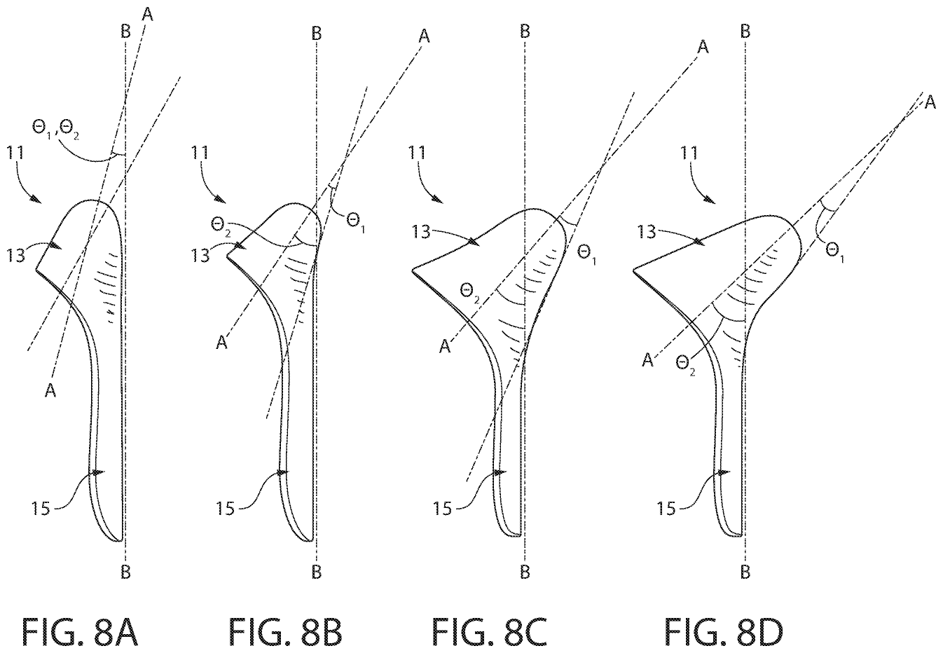

Additionally, referring now to FIGS. 8A-8D, demonstrate the change in geometry for the ear cleaning apparatus for various cone angles Ø₁ and grip angles Ø₂. As demonstrated in FIG. 8A the cone angles Ø₁ and grip angles Ø₂ may be substantially equal. As demonstrated in FIG. 8B, the cone angles Ø₁ may be about 35°, whereby the grip angle Ø₂ is greater than the cone angle Ø₁. As demonstrated in FIG. 8C, the cone angles Ø₁ may be about 40°, whereby the grip angle Ø₂ is greater than the cone angle Ø₁. As demonstrated in FIG. 8D, the cone angles Ø₁ may be about 45°, whereby the grip angle Ø₂ is greater than the cone angle Ø₁.

The ear cleaning apparatus 11 of the present invention may be formed from a single or multi-layer construction formed from material that may include cotton (e.g., non-woven or woven fabric); non-woven synthetic fiber (e.g., polypropylene, nylon, and other similar materials); other organic fibers (e.g., bamboo, hemp, modal, and other plant-based fibers), bond paper (e.g., thin card or heavy paper); formed paper pulp; thermoformed plastic (e.g., styrene, polyethylene terephthalate, polyurethane, and other similar materials); thermoformed foamed rubber (e.g., ethylene vinyl acetate, polyvinyl chloride, and other similar materials); molded plastic (e.g., polypropylene, acrylonitrile butadiene styrene, and other similar materials). The fibers may be quilted to form a macroscopic texture.

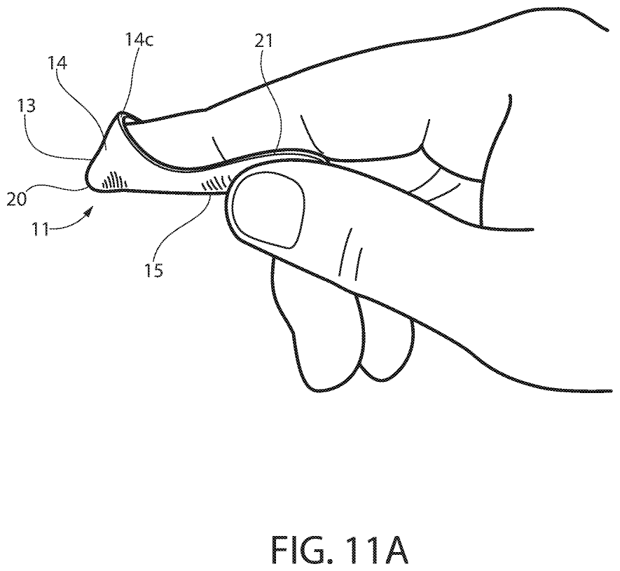

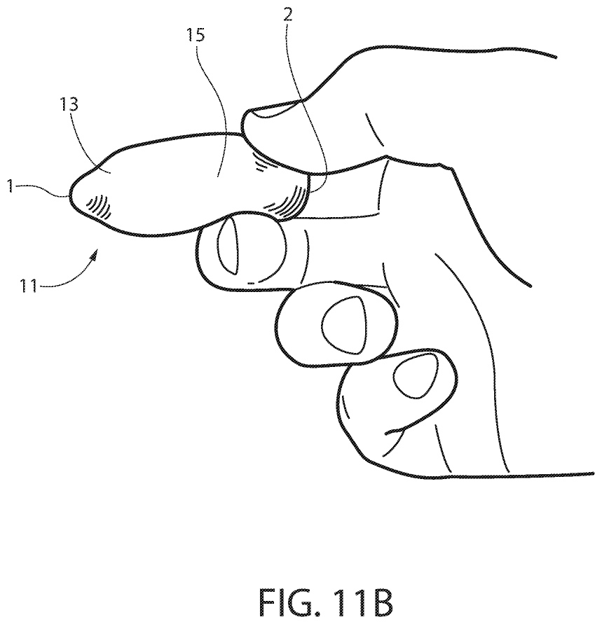

Referring now to FIGS. 10A-11B, a multi-layered ear cleaning apparatus 211 of the present invention will be discussed in greater detail. The ear cleaning apparatus 211 is similar to ear cleaning apparatus 11 except as described herein below. The description of the ear cleaning apparatus 11 above generally applies to the ear cleaning apparatus 211 described below except with regard to the differences specifically noted below. A similar numbering scheme will be used for the ear cleaning apparatus 211 as with the ear cleaning apparatus 11 except that the 200-series of numbers will be used.

The ear cleaning apparatus 211 may comprise at least two layers 267, 269 that include an outer layer 267 and an inner layer 269. The outer layer 267 may form the outer surface 211b of the ear cleaning device 211 and the inner layer 269 may form the inner surface 211a of the ear cleaning device. The edge 211c of the ear cleaning device 211 may comprise both the outer layer 267 and the inner layer 269.

The outer layer 267 may be formed of cleaning surface materials, and the inner layer 269 formed of materials which provide desired structure and/or rigidity to the ear cleaning apparatus 211. The inner layer 269 may further be referred to as the “structural layer.” The outer layer 267 and the inner layer 26 may be directly or indirectly coupled or bonded together in any appropriate manner for the materials used for the respective layers. In certain embodiments, the inner layer 269 may be included for additional purposes, such to provide a scrubbing surface, which is characterized by being a coarser and/or less flexible which is also non-absorbent. In contrast, the outer layer 267 is provided and intended primarily for a soft wiping surface which may be used dry or wet, and when used wet, the material is able to absorb moisture for enhanced wiping/cleaning. The differences between a scrubbing surface and a wiping surface are illustrated more in some of the embodiments described below.

As shown in FIG. 10B, the multi-layer construction of the ear cleaning apparatus 211 may include three layers, 267, 268, 269. These three layers include the inner layer 267, the outer layer 269, as well as an intermediate layer 268 positioned there-between. The intermediate layer 43 may be a barrier layer and formed of materials which prevent moisture penetration and/or evaporation from passing between the inner and outer surfaces 211a, 211b of the ear cleaning apparatus 211. The inner layer 269, intermediate layer 268, and outer layer 267 may be directly or indirectly coupled or bonded together in any appropriate manner for the materials used for the respective layers.

Non-limiting examples of material suitable as the outer layer 267 of the ear cleaning apparatus 211 may include: cotton (e.g., non-woven or woven fabric); non-woven synthetic fiber (e.g., polypropylene, nylon, rayon, and other similar materials); other organic fibers (e.g., bamboo, hemp, modal, and other plant-based fibers). Other types of material may also be used for the outer layer 267. The fibers may be formed by melt-blowing or as spun-lace. The melt-blown fiber may have a weight ranging from about 20 g/m to about 40 g/m—including all weight and sub-ranges there-between. The spun-lace fiber may have a weight ranging from about 40 g/m to about 80 g/m—including all weight and sub-ranges there-between. In a preferred embodiment, the fiber is spun-lace fiber.

For the intended use of cleaning the ear canal and other parts of the ear, it is generally desirable to have the material for the outer layer of a fingertip cleaner selected for absorbency and softness.

Non-limiting examples of materials suitable as the inner layer 267 of the ear cleaning apparatus 211 include: bond paper (e.g., thin card or heavy paper); formed paper pulp; thermoformed plastic (e.g., styrene, polyethylene terephthalate, and other similar materials); thermoformed foamed rubber (e.g., ethylene vinyl acetate, polyvinyl chloride, and other similar materials); molded and fibrous plastic (e.g., polypropylene, polyurethane, acrylonitrile butadiene styrene, and other similar materials).

A non-limiting example of a material suitable as the inner layer 267 include non-woven fabric formed from spun-bonded polypropylene having a weight ranging from about 15 g/m to about 60 g/m—including all weight and sub-ranges there-between. Another non-limiting example of a material suitable as the inner layer 267 include non-woven fabric formed from spun-bonded polypropylene that is coated with polyethylene having a weight ranging from about 90 g/m to about 115 g/m—including all weight and sub-ranges there-between.

Another non-limiting example of a material suitable as the inner layer 267 includes thermoplastic elastomer film. The film is continuous and is suitable as a moisture barrier. Another non-limiting example of a material suitable as the inner layer 267 includes polyurethane film. The film is continuous and is suitable as a moisture barrier while still being air-permeable.

Other types of material may also be used for the inner layer 269. For the intended use of an ear cleaning apparatus 211, it is generally desirable to have the material for the inner layer 269 selected to provide a desired amount of stiffness and/or to provide a desired texture which may be used for scrubbing. In certain embodiments, and by selection of an appropriate material, the inner layer 269 may also serve as a barrier layer.

Non-limiting examples of material suitable as the barrier layer of a multi-layered ear cleaning apparatus 211 include: thermoplastic elastomer (e.g., thermoplastic elastomers, rubber, and other similar materials); extruded film (e.g., low density polyethylene, polyvinyl chloride, and other similar materials). Other types of material may also be used for the barrier layer.

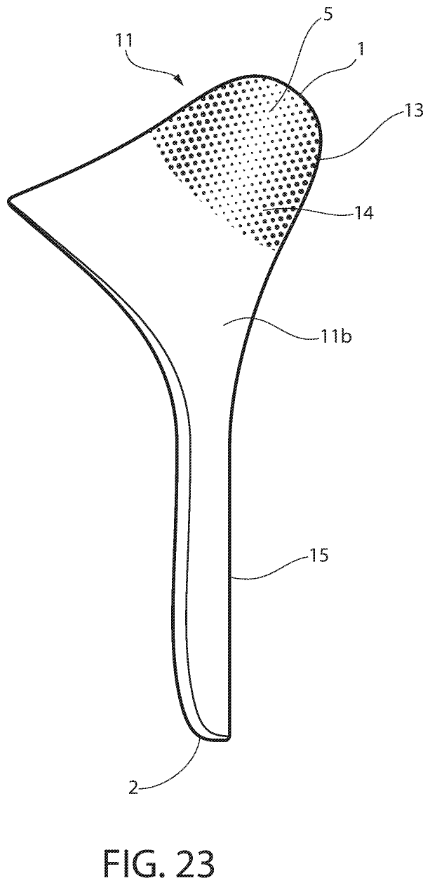

Referring now to FIG. 23, the ear cleaning apparatus of the present invention may have a textured surface 5 on the fingertip receiving portion 13, whereby the texture surface 5 is present on the outer surface 11b of ear cleaning device 11. The textured surface 5 may be imparted to the outer surface 11b of the ear cleaning apparatus 11 on the fingertip receiving portion 13 as part of the manufacturing process. The texture may have any desirable pattern—including a plurality of columns, dots, or pyramids that impart the textured surface. The texture 5 of the fingertip receiving portion 13 is a macro structure texture 5 which overlays, and is independent of, the micro structure texture of the material used to construct the fingertip receiving portion 13. For example, the macro structure texture is shown as a plurality of dimples, whereas for an fingertip receiving portion 13 formed out of non-woven cotton, the micro structure texture results from the cotton fibers themselves.

Referring now to FIGS. 11A and 11B, the fingertip receiving portion 13 is formed to fit at least partially over the fingertip to cover the tip of the fingernail. Specifically, the fingernail is inserted into the cavity 16 of the fingertip receiving portion 13. Covering the fingernail is important when cleaning the outer ear canal to avoid unwanted scratches within the ear canal which might be caused by the fingernail. In certain embodiments, the fingertip receiving portion 13 may cover just the tip of the fingernail, and in certain other embodiments, the fingertip receiving portion 13 may cover more of the fingernail, up to the entire fingernail. In certain other embodiments, the fingertip receiving portion 13 may cover even more of the fingertip, up to about the first knuckle. By covering only part of the fingertip, the ear cleaning apparatus 11 is easier to place on and remove from the finger. Also, this reduces the amount of material needed to construct the ear cleaning apparatus 11.

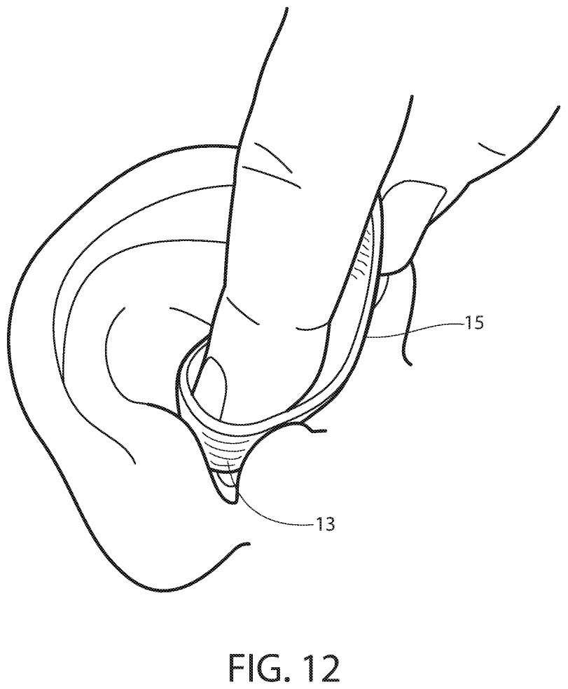

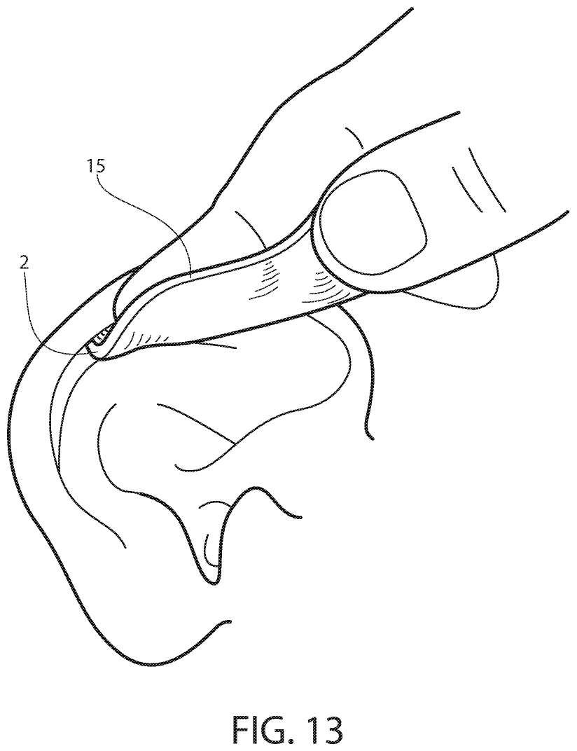

When the ear cleaning apparatus 11 is seated on the forefinger of a user, the gripping portion 15 extends down the finger to at least past the first knuckle. In certain embodiments, the gripping portion 15 may extend to between the first knuckle and up to and including the second knuckle. In certain other embodiments, the gripping portion 15 may extend beyond the second knuckle. When the fingertip receiving portion 13 is seated on the tip of a user's index finger, the user may grip the gripping portion 15 between the thumb and index finger, as shown in FIG. 12A. Alternatively, the user may choose to grip the gripping portion 15 using both the thumb and the middle finger pressed against the index finger, as shown in FIG. 12B. The overall width W₂ of the gripping portion 15 is such that it covers at least one side of the typical user's index finger. This may mean that the ear cleaning apparatus 11 may be produced in different sizes to accommodate users' different sizes of fingers.

Referring now to FIG. 12, the ear cleaning apparatus 11 may have the fingertip receiving portion 13 placed over the tip of a user's index finger, with the gripping portion 15 gripped between the thumb and index finger. Specifically, the user may insert at least a portion of a fingertip of a first finger into the cavity 16 of the ear cleaning apparatus 11 comprising the fingertip receiving portion 13 and the gripping portion 15 so that: (1) the fingertip receiving portion 13 protrudes from the fingertip and extends along the cone axis A-A that is inclined relative a cone axis B-B (also referred to as the “fingertip axis”) along which the fingertip of the first finger extends; and (2) the gripping portion 15 extends adjacent an outer surface of the fingertip of the first finger; and inserting the fingertip receiving portion 13 of the ear cleaning device 11 into the ear canal. Additionally, the user grasps the gripping portion 15 of the ear cleaning apparatus 11 between the surface of the fingertip of the first finger and a surface of a second finger during said inserting.

During use, the user straightens their index finger and positions it to be approximately axially aligned with the ear canal, i.e., and axis of the straightened index finger is spatially near and approximately parallel to an axis of the ear canal, with the fingertip receiving portion 13 placed within the outer ear canal. With the ear cleaning apparatus in position and the user having their fingers positioned thusly, the user may rotate their hand about the axis of the straightened index finger to clean the outer ear canal with the rotating motion imparted to the fingertip receiving portion 13. The fingertip receiving portion 13 will extend into the ear canal only slightly further than the tip of the user's index finger. This provides a safe method for ear cleaning. As will be discussed in connection with other embodiments below, the fingertip receiving portion 13 may be constructed with different degrees of stiffness. Embodiments which include a more flexible fingertip receiving portion 13, such as one that is formed solely from a woven material, will have limited reach into the ear canal beyond the tip of the user's finger. In contrast, embodiments which include an fingertip receiving portion 13 formed from a less flexible material will be able to reach further into the ear canal for purposes of cleaning. However, in such embodiments, the shape of the fingertip receiving portion 13 is such that the rounded end of the fingertip receiving portion 13 cannot reach the eardrum for the typical user when the ear cleaning apparatus 11 is used as described herein.

FIG. 13 illustrates a second use for the ear cleaning apparatus 11. In this use, the gripping portion 15 may be gripped with the proximal end 2 toward to tip of the index finger so that the surface of the gripping portion 15 may be used to wipe the auricle part of the ear. This may be done to help remove any dirt or waxy residue which may have migrated from the outer ear canal onto the auricle.

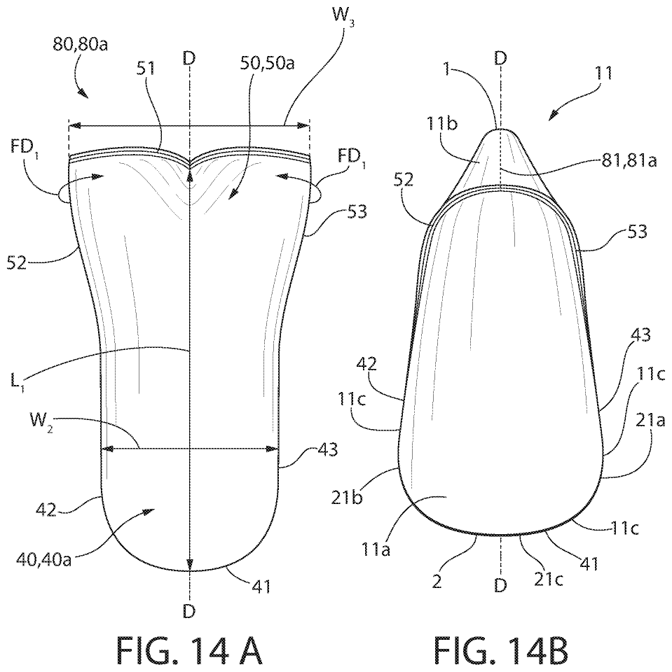

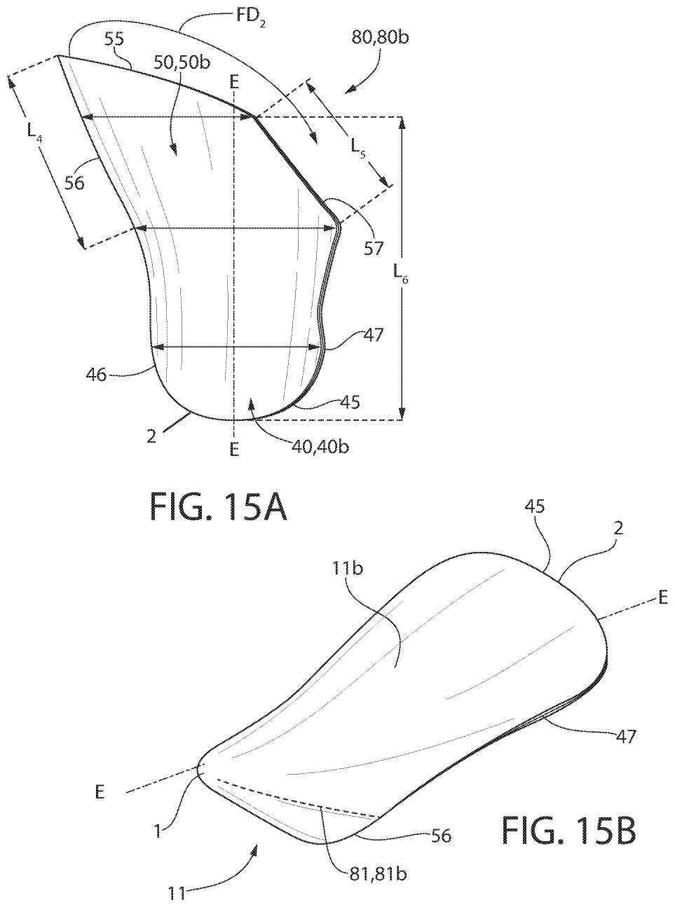

Referring now to FIGS. 14A, 14B, 15A, and 15B, the ear cleaning apparatus 11 may comprise a flexible sheet 80, 80a, 80b that is folded into a three-dimensional configuration that forms the fingertip receiving portion 13 and the gripping portion 15. In some embodiments, the fingertip receiving portion 13 and the gripping portion 15 are formed from a single flexible sheet 80, 80a, 80b that is folded into the ear cleaning apparatus 11. The ear cleaning apparatus 11 maintains the three-dimensional configuration even in an unused state when the cavity 16 is empty. The flexible sheet 80 (also referred to as a “sheet blank”) may comprise a grip blank portion 40 and a fingertip receiving blank portion 50 that is adjacent to the grip blank portion 40.

Referring now to FIGS. 14A and 14B, a first embodiment of the flexible sheet 80, 80a, the grip blank portion 40, 40a may comprises a grip blank lower edge 41 as well as a first grip blank side edge 42 that is opposite a second grip blank side edge 43. The first grip blank side edge 42 extends upward from the grip blank lower edge 41. The second grip blank side edge 43 extends upward from the grip blank lower edge 41. The grip blank lower edge 41 may be convex or flat in shape. The grip blank portion 40, 40a may have a width W₂ that is substantially equal to the width W₂ of the flap 18 of the ear cleaning apparatus 11.

According to the first embodiment of the flexible sheet 80, 80a, the fingertip receiving blank portion 50, 50a may comprise a first fingertip receiving blank side edge 52 opposite a second fingertip receiving blank side edge 53. The first fingertip receiving blank side edge 52 may extend upward and outward from the first grip blank side edge 42. The second fingertip receiving blank side edge 53 may extend upward and outward from the second grip blank side edge 43. The fingertip receiving blank portion 50, 50a may further comprise a fingertip receiving blank top edge 51 that extends between the first and second fingertip receiving blank side edges 52, 53. The fingertip receiving blank portion 50, 50a may have a maximum width W₃ that is greater than the width W₂ of the flap 18 of the ear cleaning apparatus 11.

The flexible sheet 80, 80a may further comprise a central blank axis D-D. The central blank axis D-D may intersect both the grip blank lower edge 41 and the fingertip receiving blank top edge 51. The flexible sheet 80, 80a may be symmetric about the central blank axis D-D. The grip blank lower edge 41 may be symmetrical about the central blank axis D-D. The grip blank lower edge 41 may extend between the first and second grip blank side edges 42, 43 in a direction that is substantially perpendicular to the central blank axis D-D. The fingertip receiving blank top edge 51 may be symmetrical about the central blank axis D-D. The fingertip receiving blank top edge 51 may extend between the first and second fingertip receiving blank side edges 52, 53 in a direction that is substantially perpendicular to the central blank axis D-D.

The flexible sheet 80, 80a may have a length L₁ that measured along a direction that is substantially parallel to the central axis C-C and substantially equal to the length L₁ of the ear cleaning apparatus 11.

The first grip blank side edge 42 and the second grip blank side edge 43 may have an edge profile that is mirrored about the central blank axis D-D. The first fingertip receiving blank side edge 52 and the second fingertip receiving blank side edge 53 may have an edge profile that is mirrored about the central blank axis D-D.

The first grip blank side edge 42 may extend between the grip blank lower edge 41 and the first fingertip receiving blank side edge 52 such that the first grip blank side edge 42 forms a linear or curved profile. The second grip blank side edge 43 may extend between the grip blank lower edge 41 and the second fingertip receiving blank side edge 53 such that the second grip blank side edge 43 forms a linear or curved profile.

The first fingertip receiving blank side edge 52 may extend between the first grip blank side edge 42 and the fingertip receiving blank top edge 51 such that the first fingertip receiving blank side edge 52 forms a linear or curved profile. The second fingertip receiving blank side edge 53 may extend between the second grip blank side edge 43 and the fingertip receiving blank top edge 51 such that the second fingertip receiving blank side edge 53 forms a linear or curved profile.

The first fingertip receiving blank side edge 52 may diverge from the central blank axis D-D with increasing distance from first grip blank side edge 43. The second fingertip receiving blank side edge 53 may diverge from the central blank axis D-D with increasing distance from second grip blank side edge 43. The first fingertip receiving blank side edge 52 may diverge from the central blank axis D-D when measured along a direction parallel to the central axis C-C and extending from the grip blank lower edge 41 to the fingertip receiving blank top edge 51. The second ear linsertion blank side edge 53 may diverge from the central blank axis D-D when measured along a direction extending from the grip blank lower edge 41 to the fingertip receiving blank top edge 51.

The first grip blank side edge 42 may be substantially parallel to the central blank axis D-D. The second grip blank side edge 43 may be substantially parallel to the central blank axis D-D. In other embodiments each of the first and second grip blank side edges 42, 43 may independently diverge from central blank axis D-D with increasing distance from the grip blank lower edge 41 toward the fingertip receiving blank top edge 51. In such embodiments, the first and second grip blank side edges 42, 43 may diverge from central blank axis D-D at an angle that is less than the angle at which each of the first and second fingertip receiving blank side edges 52, 53 diverge from the central blank axis D-D.

Referring now to FIGS. 15A and 15B, a second embodiment of the flexible sheet 80, 80b, the grip blank portion 40, 40b may comprises a grip blank lower edge 45 as well as a first grip blank side edge 46 that is opposite a second grip blank side edge 47. The first grip blank side edge 46 extends upward from the grip blank lower edge 45. The second grip blank side edge 47 extends upward from the grip blank lower edge 45. The grip blank lower edge 45 may be convex or flat in shape.

According to the second embodiment of the flexible sheet 80, 80b, the fingertip receiving blank portion 50, 50b may comprise a first fingertip receiving blank side edge 56 opposite a second fingertip receiving blank side edge 57. The first fingertip receiving blank side edge 56 may extend upward and outward from the first grip blank side edge 46. The second fingertip receiving blank side edge 57 may extend upward and inward from the second grip blank side edge 47. The fingertip receiving blank portion 50, 50a may further comprise an fingertip receiving blank top edge 55 that extends between the first and second fingertip receiving blank side edges 56, 57.

The flexible sheet 80, 80b may further comprise a central blank axis E-E. The central blank axis E-E may intersect both the grip blank lower edge 45 and the fingertip receiving blank top edge 55. The flexible sheet 80, 80b may be asymmetric about the central blank axis E-E. The grip blank lower edge 45 may be asymmetrical or symmetrical about the central blank axis E-E. The fingertip receiving top edge 55 may be asymmetrical about the central blank axis E-E. The first fingertip receiving blank side edge 56 and the fingertip receiving top edge 55 may be located on the same side of the central blank axis E-E.

The grip blank lower edge 45 may extend between the first and second grip blank side edges 46, 47 in a direction that is substantially perpendicular to the central blank axis E-E. The fingertip receiving blank top edge 55 may extend between the first and second fingertip receiving blank side edges 56, 57 in a direction that is substantially oblique to the central blank axis E-E. The fingertip receiving blank top edge 55 may be inclined relative to the central blank axis E-E such that the fingertip receiving blank top edge 55 extends away from the grip blank lower edge 45 as the fingertip receiving blank top edge 55 approaches the central blank axis E-E.

The first fingertip receiving blank side edge 56 has a length L₄ as measured between the fingertip receiving blank top edge 55 and the first grip blank side edge 46. The length L₄ of the first fingertip receiving blank side edge 56 may range from about 25 mm to about 35 mm. The length L₄ of the first fingertip receiving blank side edge 56 may be about 36 mm. The second fingertip receiving blank side edge 57 has a length L₅ as measured between the fingertip receiving blank top edge 55 and the second grip blank side edge 47. The length L₅ of the second fingertip receiving blank side edge 57 may range from about 15 mm to about 25 mm. The length L₄ of the first fingertip receiving blank side edge 56 may be about 25 mm.

According to the second embodiment of the flexible sheet 80, 80b, the length L₄ of the first fingertip receiving blank side edge 56 is greater than the length L₅ of the second fingertip receiving blank side edge 57. A ratio of the length L₄ of the first fingertip receiving blank side edge 56 to the length L₅ of the second fingertip receiving blank side edge 57 may range from about 10.0:1.0 to about 1.1:1—including all ratios and sub-ranges there-between. In a preferred embodiment, the ratio of the length L₄ of the first fingertip receiving blank side edge 56 to the length L₅ of the second fingertip receiving blank side edge 57 may range from about 3.0:1.0 to about 1.5:1—including all ratios and sub-ranges there-between.

The flexible sheet 80, 80b according to this embodiment may further comprise a length L₆ that extends from a transition point that exists between the insertion grip blank top edge 55 and the second fingertip receiving blank side edge 57 and the grip blank lower edge 45. The length L6 may be greater than each of the length L₄ of the first fingertip receiving blank side edge 56 and the length L₅ of the second fingertip receiving blank side edge 57.

The first grip blank edge 46 and the second grip blank edge 47 may have an edge profile that is mirrored about the central blank axis E-E. In other embodiments, the first grip blank edge 46 and the second grip blank edge 47 may have an edge profile that is not mirrored (i.e., asymmetrical) about the central blank axis E-E. The first fingertip receiving blank side edge 56 and the second fingertip receiving blank side edge 57 may have an edge profile that is not mirrored about the central blank axis E-E.

The first grip blank side edge 46 may extend between the grip blank lower edge 45 and the first fingertip receiving blank side edge 56 such that the first grip blank side edge 46 forms a linear or curved profile. The second grip blank side edge 47 may extend between the grip blank lower edge 45 and the second fingertip receiving blank side edge 57 such that the second grip blank side edge 47 forms a linear or curved profile.

The first fingertip receiving blank side edge 56 may extend between the first grip blank side edge 46 and the fingertip receiving blank top edge 55 such that the first fingertip receiving blank side edge 56 forms a linear or curved profile. The second fingertip receiving blank side edge 57 may extend between the second grip blank side edge 47 and the fingertip receiving blank top edge 55 such that the second fingertip receiving blank side edge 57 forms a linear or curved profile.

The first fingertip receiving blank side edge 56 may diverge from the central blank axis E-E with increasing distance from first grip blank side edge 46. The second fingertip receiving blank side edge 57 may converge toward the central blank axis E-E with increasing distance from second grip blank side edge 47. The first fingertip receiving blank side edge 56 may diverge from the central blank axis E-E when measured along a direction parallel to the central axis E-E and extending from the grip blank lower edge 45 to the fingertip receiving blank top edge 55. The second fingertip receiving blank side edge 57 may converge toward the central blank axis E-E when measured along a direction extending from the grip blank lower edge 45 to the fingertip receiving blank top edge 55.

The first grip blank side edge 46 may be substantially parallel to the central blank axis E-E. The second grip blank side edge 47 may be substantially parallel to the central blank axis E-E. In other embodiments each of the first and second grip blank side edges 46, 47 may independently diverge from central blank axis E-E with increasing distance from the grip blank lower edge 45 moving toward the fingertip receiving blank top edge 55. In such embodiments, the first grip blank side edge 46 may diverge from central blank axis E-E at an angle that is less than the angle at which each of the first fingertip receiving blank side edge 56 diverges from the central blank axis E-E.

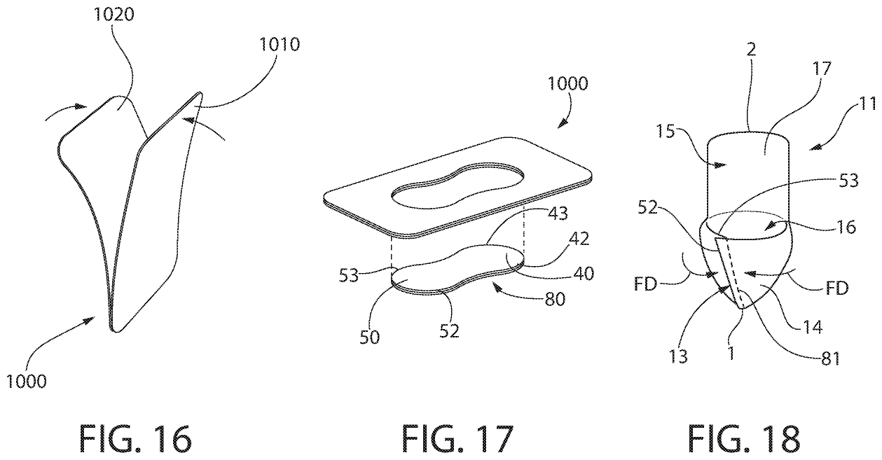

Referring now to FIGS. 16-18, the ear cleaning apparatus of the present invention may be produced from one or more laminate sheets 1000 of material—as discussed further herein. The laminate sheets 1000 may be formed by laminating together a first sheet of material 1010 and a second sheet of material 1020. The first sheet of material 1010 may be formed from the same material as the outer layer 267 of the multi-layered the ear cleaning apparatus 211. The second sheet of material 1020 may be formed from the same material as the inner layer 269 of the multilayered ear cleaning apparatus 211. Although not pictured, a third sheet of material may be positioned between the first and second sheets 1010, 1020 of material, whereby the third sheet is formed from the same material as the barrier layer 268 of the multilayered ear cleaning apparatus 211.

Referring now to FIG. 17, once the first, second and/or third sheets of material are laminated together to form the laminate sheet 1000, one or more of the sheet blanks 80 can be cut from the laminate sheet 1000. The sheet blanks 80 may be cut according to any suitable process including, but not limited to, die cutting, roll cutting, and the like. Referring now to FIG. 18, once the sheet blank 80 is cut from the laminate sheet 1000, the fingertip receiving blank portion 50 may be folded along a fold direction FD to form the fingertip receiving portion 13.

Referring now to FIGS. 14A and 14B, according to some embodiments the fingertip receiving blank portion 50a may be folded along a fold direction FD₁ such that the first fingertip receiving blank side edge 52 meets the second fingertip receiving blank side edge 53 at a distance that is substantially equidistance between the first and second fingertip receiving blank side edges 52, 53. Meeting the first and second fingertip receiving portion blank side edges 52, 53 may create a partial overlap therein.

Once folded along the fold direction FD₁, the first and second fingertip receiving blank side edges 52, 53 may be fixed relative to each other along the partial overlap. Non-limiting examples of how to fix the first and second fingertip receiving blank side edges 52, 53 along the partial overlap include adhesive bonding, stitching, ultrasonic sewing, fastener, and the like. Once fixed to each other, a seam 80, 80a may be created between the first and second fingertip receiving blank side edges 52, 53, whereby the seam 80, 80a extends along the outer surface 14b of the conical wall 14 of the fingertip receiving portion 13 of the ear cleaning apparatus 11. According to this embodiment, the seam 80, 80a may be arranged substantially symmetrically with at least one of the cone axis A-A and/or the grip axis B-B. The seam 80, 80a may extend from the apex 20 to the open end 18 of the conical wall 16.

Referring now to FIGS. 15A and 15B, according to some embodiments the fingertip receiving blank portion 50b may be folded along a fold direction FD₂ such that the first fingertip receiving blank side 56 meets the second fingertip receiving blank side edge 57 at a distance that is substantially non-equidistance between the first and second fingertip receiving blank side edges 56, 57. Meeting the first and second fingertip receiving blank side edges 56, 57 may create a partial overlap therein.

Once folded along the fold direction FD₂, the first and second fingertip receiving blank side edges 56, 57 may be fixed relative to each other along the partial overlap. Non-limiting examples of how to fix the first and second fingertip receiving blank side edges 56, 57 along the partial overlap include adhesive bonding, stitching, fastener, and the like. Once fixed to each other, a seam 80, 80b may be created between the first and second fingertip receiving blank side edges 56, 57, whereby the seam 80, 80b extends along the outer surface 14b of the conical wall 14 of the fingertip receiving portion 13 of the ear cleaning apparatus 11. According to this embodiment, the seam 80, 80b may be arranged substantially asymmetrically with respect to at least one of the cone axis A-A and/or the grip axis B-B. The seam 80, 80b may extend from the apex 20 to the open end 18 of the conical wall 16. The resulting ear cleaning apparatus 11 may comprise the first fingertip receiving blank side edge 56 forming at least a portion of the lower edge 14c of the conical wall 14. In particular, the resulting ear cleaning apparatus 11 may comprise the first fingertip receiving blank side edge 56 forming at least a portion of the lower edge 14c of the conical wall 14 that extends across opposite sides of the cone axis A-A and the grip axis B-B.

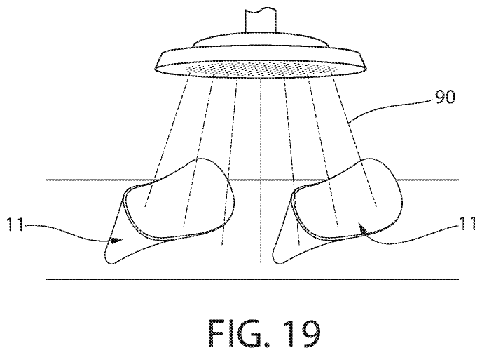

Referring now to FIG. 19, the ear cleaning apparatus 11 of the present invention may be further modified to enhance cleaning performance during use. Specifically, the ear cleaning apparatus 11 may be pre-treated with a cleaning solution 90. The cleaning solution 90 may comprise a hydrophilic component. The cleaning solution 90 may comprise an oleophilic component. The cleaning solution 90 may comprise both the hydrophilic component and the oleophilic component as a surfactant. Non-limiting examples of the surfactant include polyoxyethylene hydrogenated castor oil. The cleaning solution 90 may further comprise one or more of water, propylene glycol, methylparaben, phenoxethol, and/or sodium lactate.

The ear cleaning apparatus 11 may be pre-treated with the cleaning solution such that the outer surface 14b of the conical wall 14 comprises the cleaning solution 90. In other embodiments, other portions of the ear cleaning apparatus 11 may be pre-treated with the cleaning solution 90.

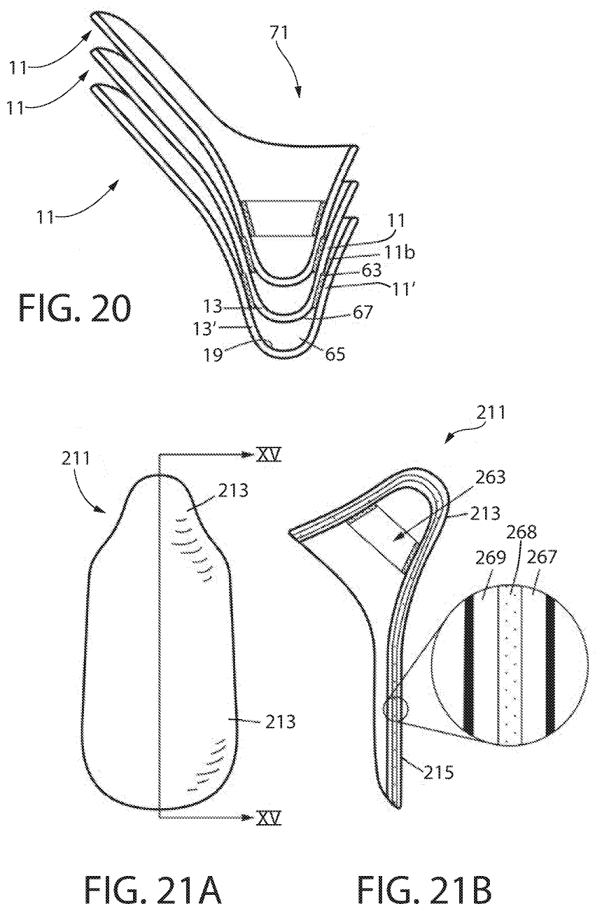

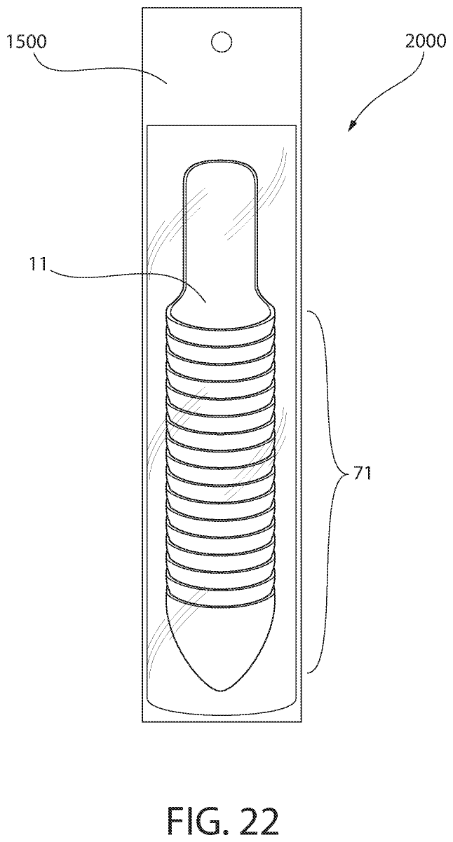

FIGS. 20 and 22 show a plurality of ear cleaning apparatuses 11 placed in a stacked arrangement 71. In this stacked arrangement, the fingertip receiving portion 13 of a first ear cleaning apparatus 11 is nested inside the cavity 16 of the conical wall 14 of the fingertip receiving portion 13′ of a second ear cleaning apparatus 11′. The outer surface 11b, specifically the outer surface 14b of the conical wall 14, presses against the inner surface 14a of the conical wall 14 to create an enclosed space 65 between the external tip 67 of the fingertip receiving portion 13 of an ear cleaning apparatus 11 and the closed end 19 of the cavity 16 of the fingertip receiving portion 13 of a second ear cleaning apparatus 11′.

Referring now to FIGS. 20-21B, liquid may be maintained within this enclosed space 65, by sufficient compression against the outer surface 14b of the conical wall 14b of the first fingertip cleaning apparatus 11 and the inner surface 14c of the second fingertip cleaning apparatus 11′. The enclosed space 65 may create a moisture tight enclosure that can prevent substantial loss of liquid that has been pre-applied to the tip 67 of the fingertip cleaning apparatus 11 due to evaporation over an extended period of time. The liquid contained in the enclosed space 65 may be the cleaning composition 90 applied during pre-treatment of the ear cleaning apparatus 11. Thus, in the stacked arrangement 71, the ear cleaning apparatus 11 may each have the external tips 67 pre-moistened with a cleaning solution 90, whereby the adjacent ear cleaning apparatus 11 helps to preserve the pre-moistened state of the nested ear cleaning apparatus 11.

In some embodiments, a barrier band 63 may be included as a lining on the inner surface 14a of the conical wall 14 of the fingertip receiving portion 13. The barrier band 63 of the second ear cleaning apparatus 11′ presses against the outer surface 11b of the first ear cleaning apparatus 11—specifically the outer surface 14b of the conical wall 14—to create an enclosed space 65 between the external tip 67 of the fingertip receiving portion 13 of an ear cleaning apparatus 11 and the closed end 19 of the cavity 16 of the fingertip receiving portion 13 of a second ear cleaning apparatus 11′. The barrier band 63 of each ear cleaning apparatus 11 may further help preserve the pre-moistened state of the nested ear cleaning apparatus 11 by further sealing the enclosed space 65 created between adjacent fingertip cleaning apparatus 11, 11′.

Referring now to FIG. 22, the plurality of ear cleaning apparatuses 11 placed in a stacked arrangement 71 may be placed into a packaging 1500, which may or may not form an air-tight seal around the plurality of ear cleaning apparatuses 11. Together, the combination of the plurality of ear cleaning apparatuses 11 contained within the packaging 1500 form a kit 2000, which may be made available for sale to consumers.

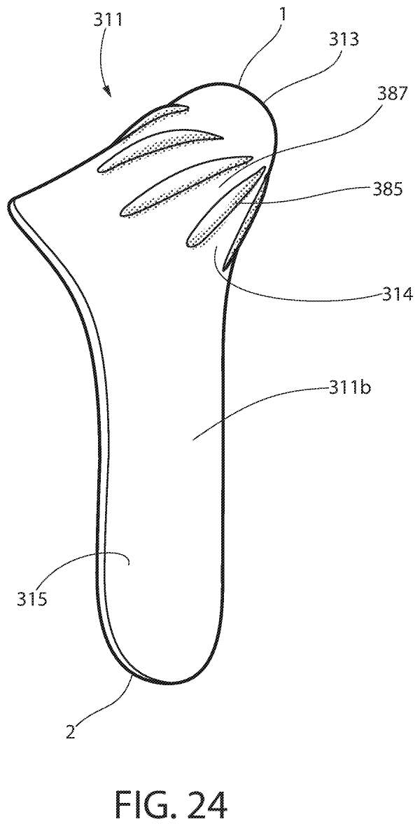

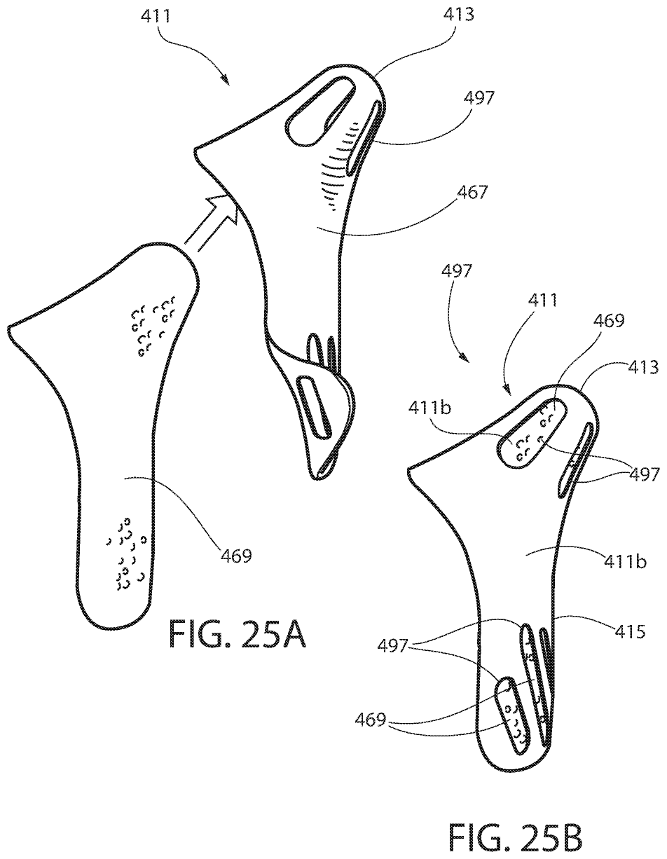

Referring now to FIGS. 24-32, additional embodiments of the ear cleaning apparatus of present invention will be provided. The description of the ear cleaning apparatus 11, 211 above generally applies to the following embodiments described below except with regard to the differences specifically noted below. A similar numbering scheme will be used for the ear cleaning apparatus 311, 411, 511, 611, 711, 811, 911, 1111, and 1211 as with the ear cleaning apparatuses 11 and 212 except that the 300-series, 400-series, 500-series, 600-series, 700-series, 800-series, 900-series, 1100-series, and 1200-series of numbers will be used.

Referring to FIG. 24 in particular, an ear cleaning apparatus 311 according to another embodiment of the present invention may comprise a texture placed on the outer surface of the fingertip receiving portion 313. In multi-layer embodiments, the texture may be formed in just the outer layer, or it may be formed in both the outer layer and any one or more underlying layers. As shown, the texture is a plurality of ridges 385 and valleys 387, both of which extend around the external surface of the fingertip receiving portion 383 so that they are non-parallel to the cone axis formed by the fingertip receiving portion 383.

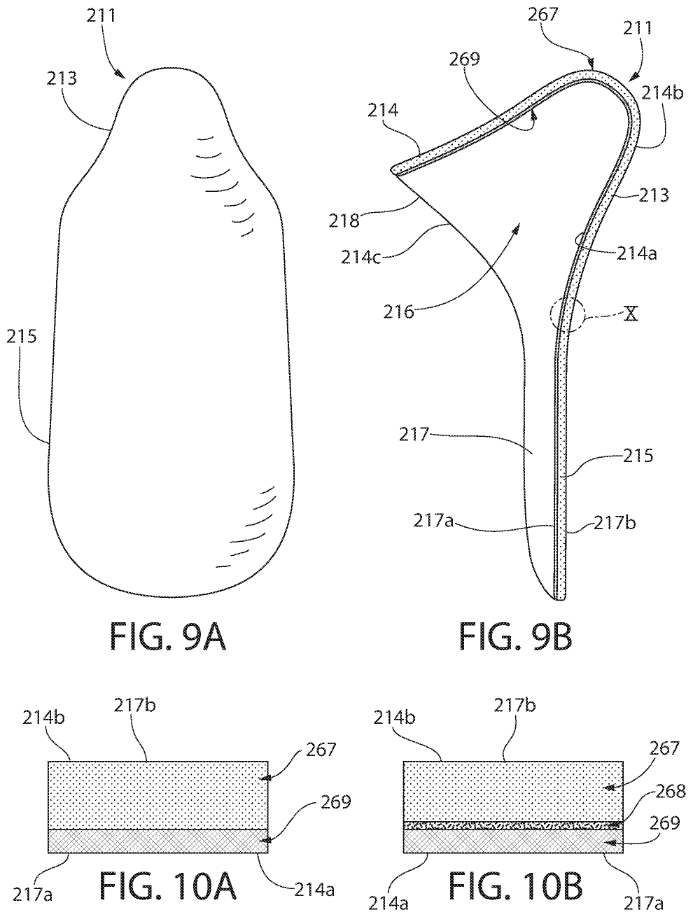

Referring to FIGS. 13A-B, an ear cleaning apparatus 411 according to another embodiment of the present invention may comprise a multi-layer construction. The overall shape of the fingertip receiving portion 413 and the gripping portion 415 of this ear cleaning apparatus 411 is similar to the ear cleaning apparatus 11 and 211 of FIGS. 1-15B. Both the fingertip receiving portion 413 and the gripping portion 415 include apertures 497 formed in the outer layer 467. Any number of apertures 497 may be included on either of the fingertip receiving portion 413 and the gripping portion 415. The apertures 497 may also have any desired geometric shape, such as the elongate shape shown, or alternatively circular, square, irregular, or any combination of shapes. When the outer layer 467 is coupled to the structural layer 469, portions of the structural layer 469 are exposed through the apertures 497. The exposed portions of the structural layer 469 may be formed as a textured surface. By forming the ear cleaning apparatus 411 in this manner, both the fingertip receiving portion 413 and the gripping portion 415 include multi-purpose cleaning surfaces which have part of the surface formed with the absorbency and softness of the outer layer 467 interspersed with a textured surface formed by the structural layer 469. Stated otherwise, the outer surface 411b of the ear cleaning apparatus may comprise both the outer layer 467 and the inner layer 469.

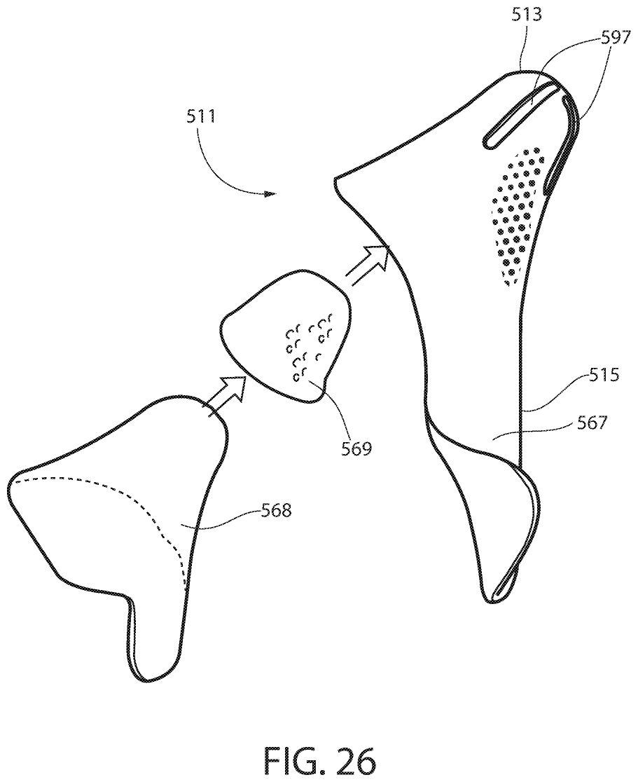

Referring now to FIG. 26, an ear cleaning apparatus 511 according to another embodiment of the present invention may comprise a multi-layer construction. The overall shape of the fingertip receiving portion 513 and the gripping portion 515 of this ear cleaning apparatus 511 is similar to the ear cleaning apparatus 11 and 211 of FIGS. 1-15B. In this ear cleaning apparatus 511, the fingertip receiving portion 513 has a multi-layer construction, while the gripping portion 515 has a single-layer construction. An outer layer 567 forms part of both the fingertip receiving portion 513 and the gripping portion 515. As part of the fingertip receiving portion 513, the outer layer 567 includes a plurality of apertures 597. The apertures 597 may have any geometrical shape, whether regular or irregular. The fingertip receiving portion 513 also includes a structural layer 569 and a barrier layer 568, with the structural layer 569 formed as a middle layer between the outer layer 567 and the barrier layer 568. The barrier layer 568 may extend at least partially into the gripping portion 515. The structural layer 569 has a textured surface, part of which is exposed through the apertures 597 formed in the outer layer 567.

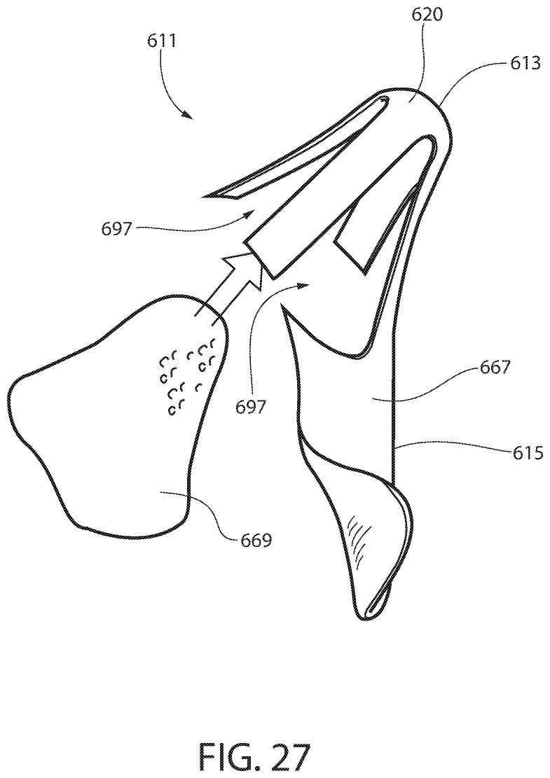

Referring now to FIG. 27, an ear cleaning apparatus 611 according to another embodiment of the present invention may comprise a multi-layer construction. The overall shape of the fingertip receiving portion 613 and the gripping portion 615 of this ear cleaning apparatus 611 is similar to the ear cleaning apparatus 11 and 211 of FIGS. 1-15B. In this ear cleaning apparatus 611, the fingertip receiving portion 613 has a multi-layer construction, while the gripping portion 615 has a single-layer construction. An outer layer 677 forms part of both the fingertip receiving portion 613 and the gripping portion 615. As part of the fingertip receiving portion 613, the outer layer 667 includes a plurality of slots 697 extending from an edge of the fingertip receiving portion 613 toward the tip 620. The slots 697 may have any geometrical shape, whether regular or irregular. A structural layer 669 also forms part of the fingertip receiving portion 613. The structural layer 669 has a textured surface, part of which is exposed through the slots 697 formed in the outer layer 667.

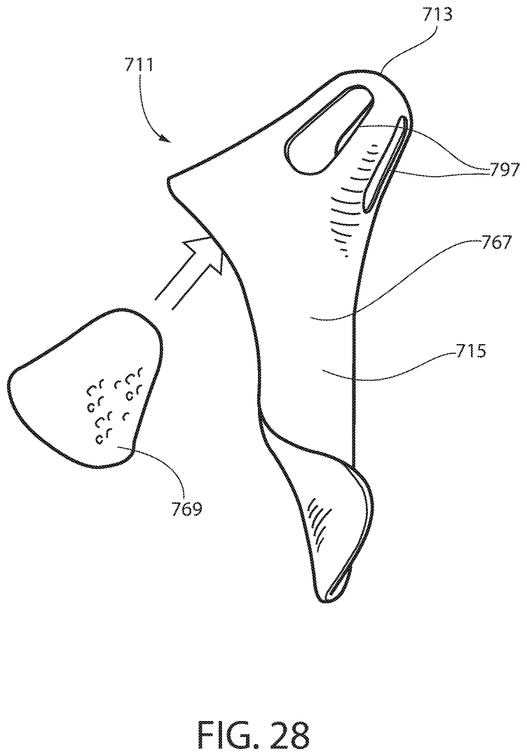

Referring now to FIG. 28, an ear cleaning apparatus 711 according to another embodiment of the present invention may comprise a multi-layer construction. The overall shape of the fingertip receiving portion 713 and the gripping portion 715 of this ear cleaning apparatus 711 is similar to the ear cleaning apparatus 11, 211 of FIGS. 1-15B. In this cleaning apparatus 711, the fingertip receiving portion 713 has a multi-layer construction, while the gripping portion 715 has a single-layer construction. An outer layer 767 forms part of both the fingertip receiving portion 713 and the gripping portion 715. As part of the fingertip receiving portion 713, the outer layer 767 includes a plurality of apertures 797. The apertures 797 may have any geometrical shape, whether regular or irregular. A structural layer 769 also forms part of the fingertip receiving portion 713. The structural layer 769 has a textured surface, part of which is exposed through the apertures 797 formed in the outer layer 767.

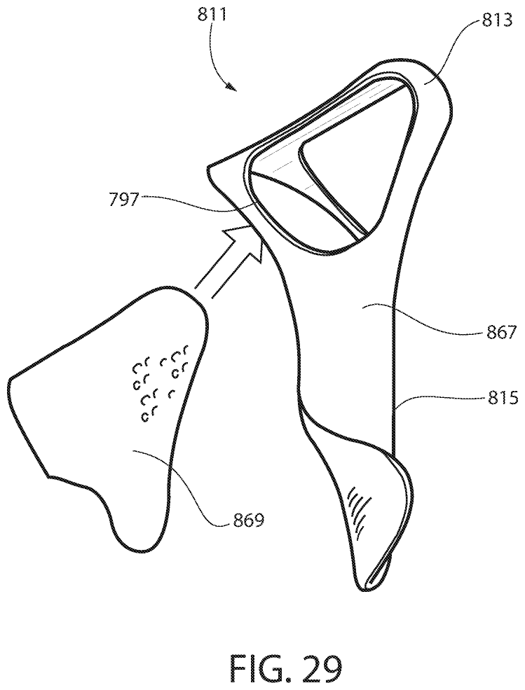

Referring now to FIG. 29, an ear cleaning apparatus 811 according to another embodiment of the present invention may comprise a multi-layer construction. The overall shape of the fingertip receiving portion 813 and the gripping portion 815 of this cleaning apparatus 811 is similar to the cleaning apparatus 11, 211 of FIGS. 1-15B. In this cleaning apparatus 811, the fingertip receiving portion 813 has a multi-layer construction, while the gripping portion 815 has a single-layer construction. An outer layer 867 forms part of both the fingertip receiving portion 813 and the gripping portion 815. As part of the fingertip receiving portion 813, the outer layer 867 includes two apertures 897, each being on opposite sides of the fingertip receiving portion 813 from the other. Strips of the outer layer 867 are therefore formed between the two apertures 897. The apertures 897 may have any geometrical shape, whether regular or irregular. A structural layer 869 also forms part of the fingertip receiving portion 813. The structural layer 869 has a textured surface, part of which is exposed through the apertures 897 formed in the outer layer 867.

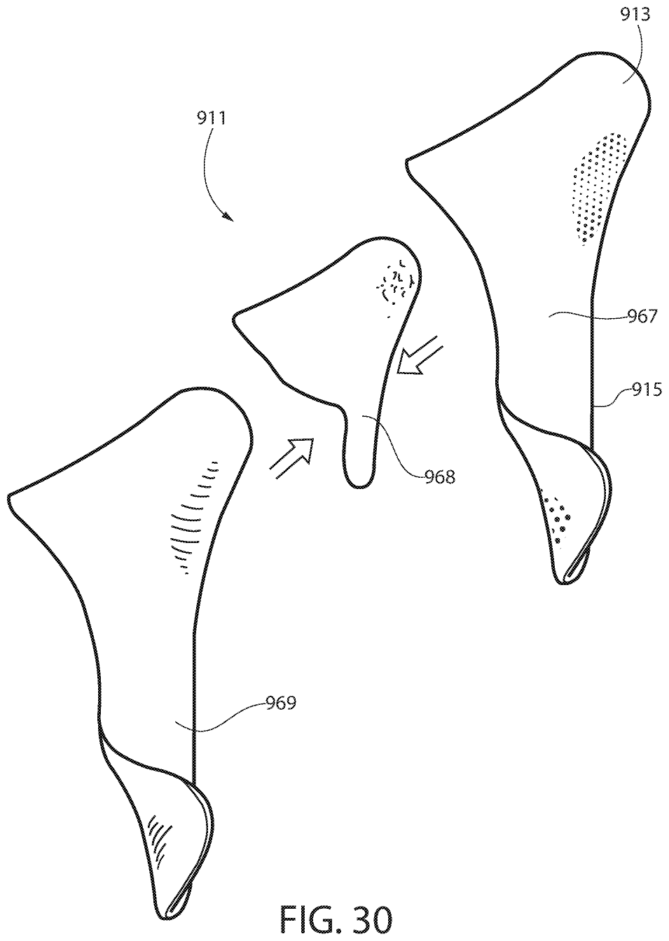

Referring now to FIG. 30, an ear cleaning apparatus 911 according to another embodiment of the present invention comprises a multi-layer construction. The overall shape of the fingertip receiving portion 913 and the gripping portion 915 of this cleaning apparatus 911 is similar to the cleaning apparatus 11, 211 of FIGS. 1-15B. In this cleaning apparatus 911, the fingertip receiving portion 913 has a three-layer construction, while the gripping portion 915 has a two-layer construction. The ear cleaning apparatus 911 is formed by an outer layer 967, a barrier layer 968, and a structural layer 969. The outer layer 967 and the structural layer 969 form part of both the fingertip receiving portion 913 and the gripping portion 915, while the barrier layer 968 forms only part of the fingertip receiving portion 913.

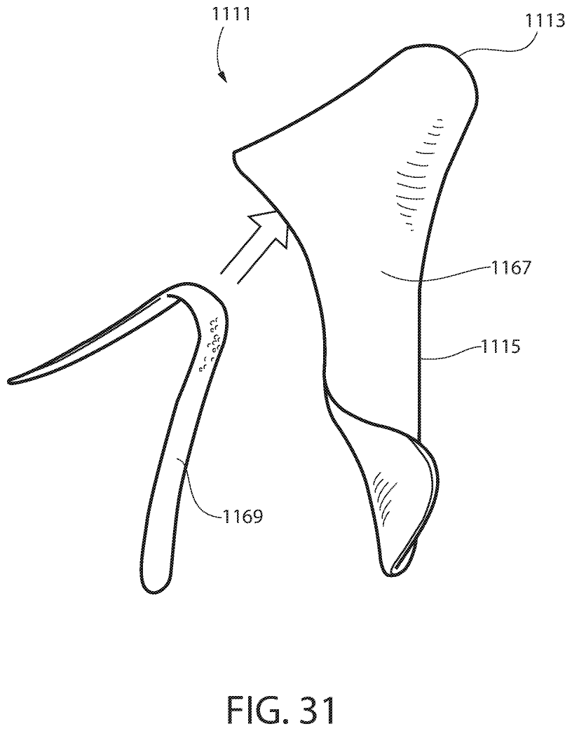

Referring now to FIG. 31, an ear cleaning apparatus 1111 according to another embodiment comprises a multi-layer construction. The overall shape of the fingertip receiving portion 1113 and the gripping portion 1115 of this ear cleaning apparatus 1111 is similar to the ear cleaning apparatus 11, 211 of FIGS. 1-15B. In this ear cleaning apparatus 1111, the fingertip receiving portion 1113 has a two-layer construction, while the gripping portion 1115 has a single-layer construction. An outer layer 1167 forms part of both the fingertip receiving portion 1113 and the gripping portion 1115. The fingertip receiving portion 1113 includes a structural layer 1169 formed as a strip of material which extends along two sides and the tip of the fingertip receiving portion 1113. By forming the structural layer 1169 as a strip of material, some of the advantages of having the structural layer 1169 may still be realized, such as providing some structure to the outer layer 1167, while also reducing manufacturing costs by reducing the amount of material needed to form the structural layer 1169.

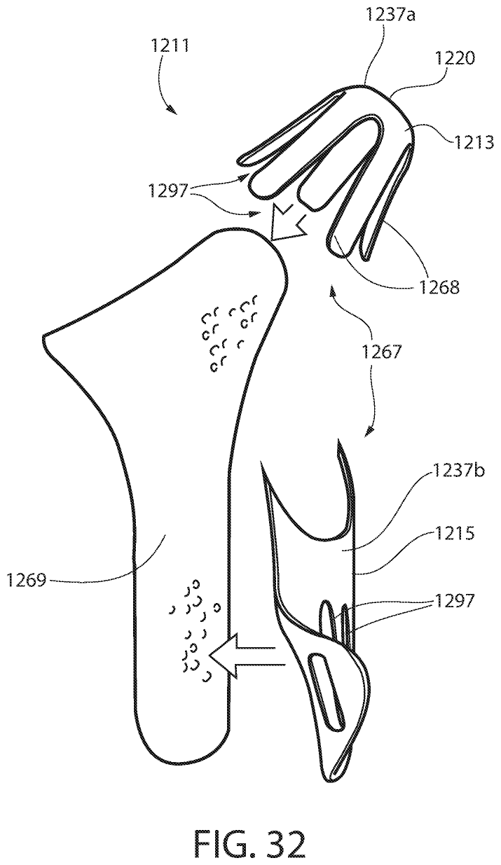

Referring now to FIG. 32, an ear cleaning apparatus 1211 according to another embodiment comprises a multi-layer construction. The overall shape of the fingertip receiving portion 1213 and the gripping portion 1215 of this ear cleaning apparatus 1211 is similar to the ear cleaning apparatus 11, 211 of FIGS. 1-15B. In this ear cleaning apparatus 1211, both the fingertip receiving portion 1213 and the gripping portion 1235 have a two-layer construction, each having an outer layer 1267 and a structural layer 1269. The outer layer 1267 is formed in two separate parts, a first part 1237a for the fingertip receiving portion 1213 and a second part 1237b for the gripping portion 1215. The first part 1237a of the outer layer 1267 includes a plurality of fingers extending from the tip 1220 of the fingertip receiving portion 1213, with a space 1297 being formed between adjacent fingers. The first part 1237a may include any number of fingers and spaces 1297. The second part 1237b of the outer layer 1267 includes apertures 1297. The second part 1237a may include any number of apertures 1297, and the apertures 1297 may have any desired geometric shape or combination of shapes. With the first and second parts 1237a, 1237b of the outer layer 167 coupled to the structural layer 1269, portions of the structural layer 1269 are exposed through the spaces 1297 between the fingers of the first part 1237a and through the apertures 1297 of the second part 1237b. The exposed portions of the structural layer 1269 may be formed as a textured surface. By forming the ear cleaning apparatus 1211 in this manner, both the fingertip receiving portion 1213 and the gripping portion 1215 include multi-purpose cleaning surfaces which have part of the surface formed with the absorbency and softness of the outer layer 1267 interspersed with a textured surface formed by the structural layer 1269.

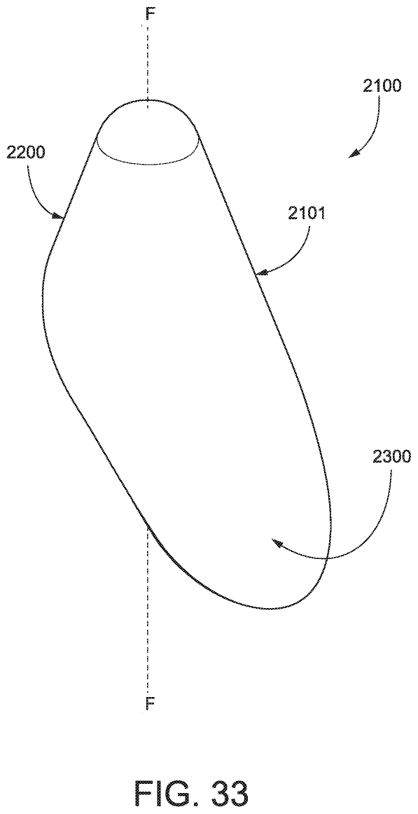

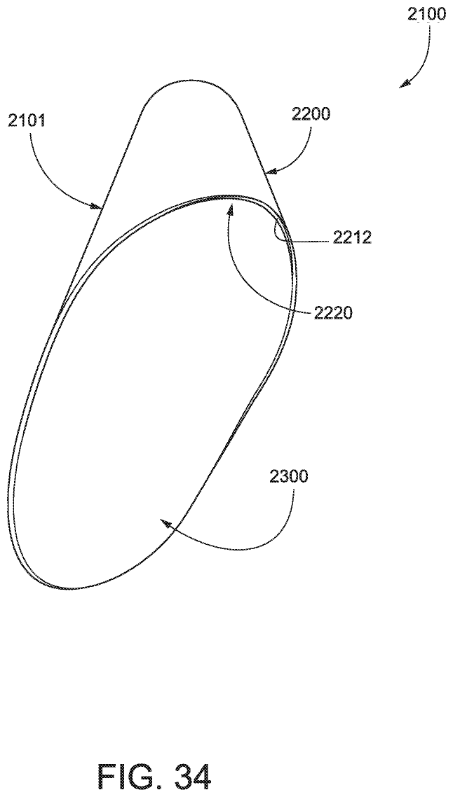

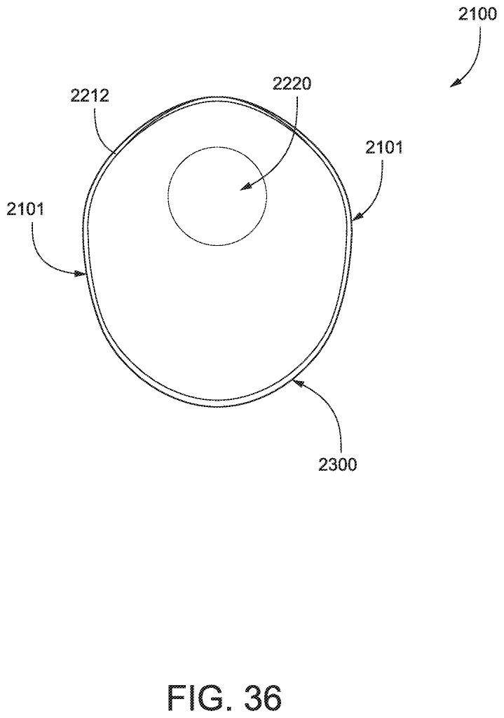

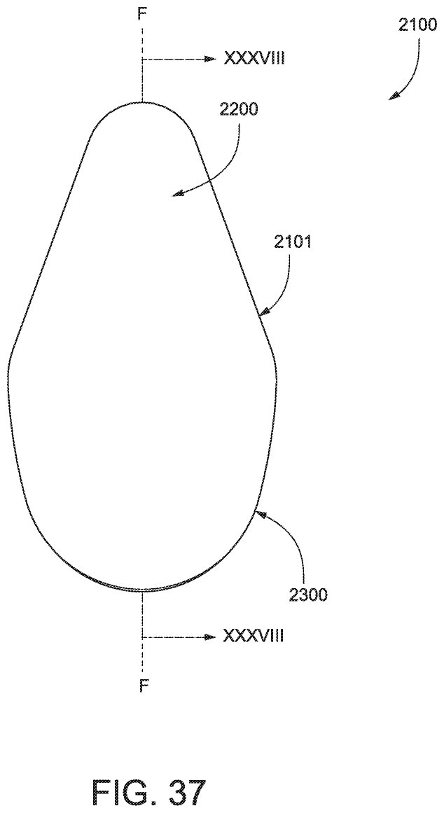

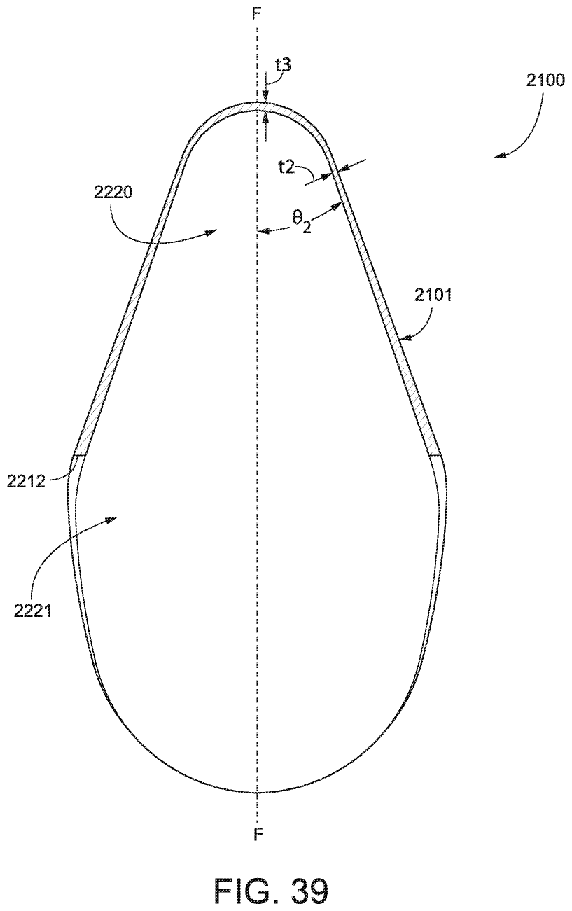

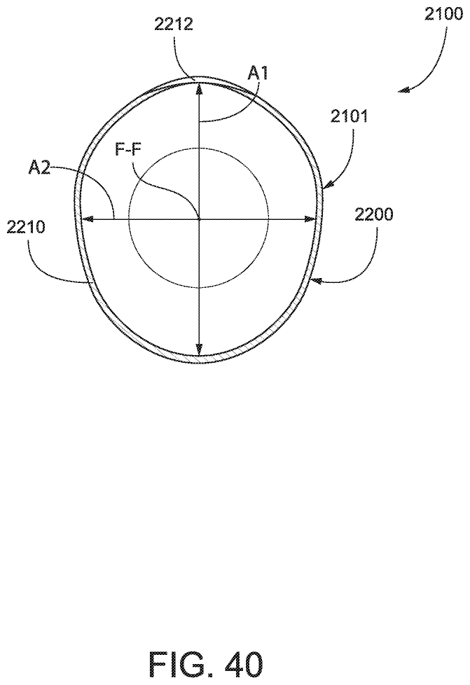

Referring now to FIGS. 33-40 concurrently, a fingertip cleaning apparatus 2100 according to another embodiment of the present invention is illustrated. The fingertip cleaning apparatus 2100 is configured to be placed over a user's index finger for cleaning earwax, dirt, and other unwanted contaminants from various external body surfaces and/or body cavities. It can also be sued to remove makeup. Non-limiting examples of suitable external body surfaces include ear, nose, forehead, face, neck, armpit, and toes. Moreover, while the fingertip cleaning apparatus 2100 is exemplified as being used to clean an external body surface, it can also be used as an applicator to apply a substance to a user's skin and/or external body surface.

The fingertip cleaning apparatus 2100 takes the form of a shape-retaining three-dimensional structure 2101 that generally comprises a fingertip receiving portion 2200 and a gripping portion 2300. As will be described in greater detail below, three-dimensional structure 2101 (and thus the fingertip cleaning apparatus 2100 in certain embodiments) is press formed from a single panel of material that is press-formed to assume (and retain) the three-dimensional structure 2101. Thus, the three-dimensional structure 2101 is free of seams and has a continuous and uninterrupted outer surface.

The three-dimensional structure 2101 retains its shape in a free state (i.e., a state in which no external forces are applied to the fingertip cleaning apparatus 2100). Thought of another way, the three-dimensional structure 2101 is a self-supporting structure that maintains the three-dimensional structure/shape in the presence of gravity while laying on a horizontal surface and no other external forces are applied.

In one embodiment, the three-dimensional structure 2101 is press-formed from a single panel of material, such as fabric. In one such embodiment, the fabric is an adsorbent material. The fabric may be a fibrous fabric, such a woven fabric or a non-woven fibrous fabric. One suitable non-woven fibrous fabric is a nonwoven polyester fibrous fabric. When a flat panel of material, such as the fibrous fabric discussed above, is subjected to the compression forces of a press (discussed below) the fibers can be stretched in certain areas while at the same time undergoing substantial compaction. As a result, certain portions of the three-dimensional structure 2101 may become stiffer and form a three-dimensional conical structure that maintains its shape even after the compression forces of the press are released.

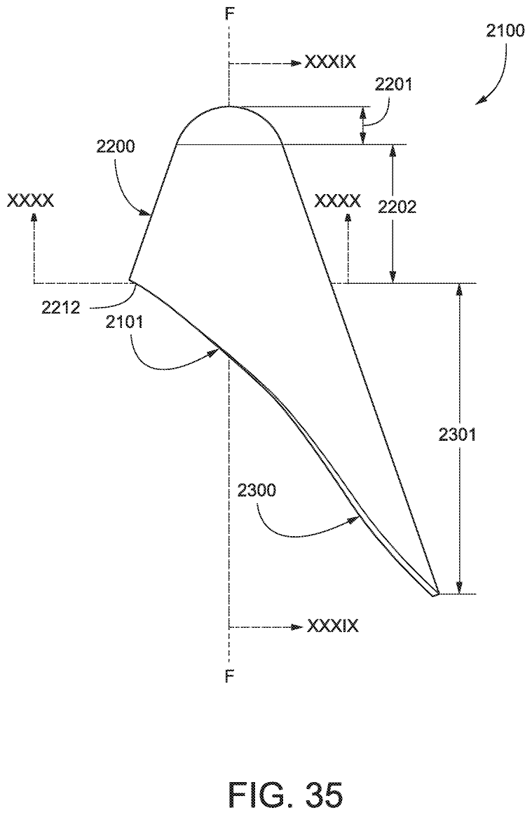

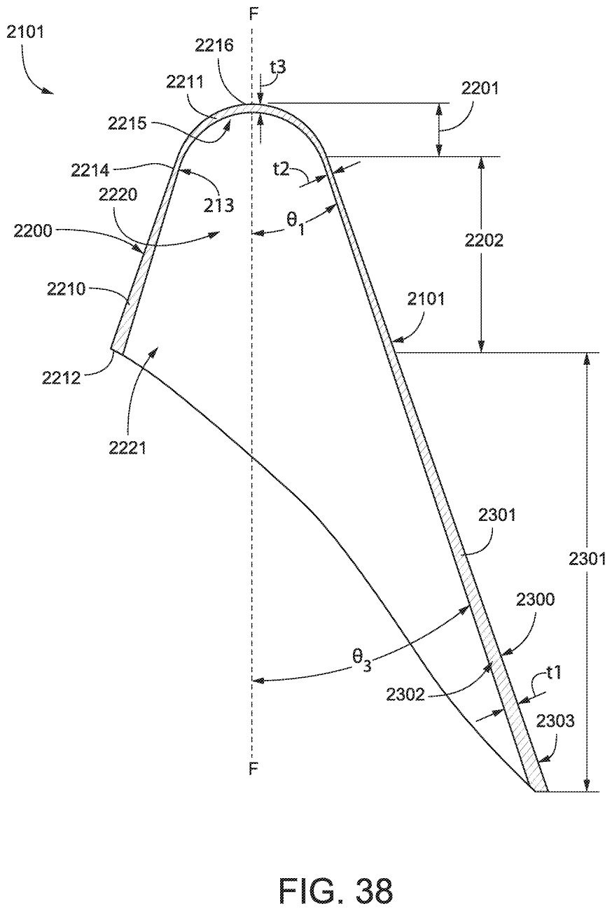

The three-dimensional structure 2101 of the fingertip cleaning apparatus 2100 is formed about and extends along a cone axis F-F. As can be seen best in FIGS. 35 and 38, the three-dimensional structure 2101 of the fingertip cleaning apparatus 2100 comprises an apex section 2201 that forms a distal-most axial section of the fingertip cleaning apparatus 2100, a flap section 2301 that forms a proximal-most axial section of the fingertip cleaning apparatus 2100, and a cone section 2202 that forms a middle axial section of the fingertip cleaning apparatus 2100 located between the apex section 2201 and the flap section 2301. The apex section 2201 and the cone section 2202 collectively define the fingertip receiving portion 2200 of the three-dimensional structure 2101 of the fingertip cleaning apparatus 2100 while the flap section 2301 defines the gripping portion 300 of the three-dimensional structure 2101 of the fingertip cleaning apparatus 2100.

The cone section 2202 of the fingertip receiving portion 2200 comprises a conical wall 2210 that surrounds the cone axis F-F. The apex section 2201 of the fingertip receiving portion 2200 comprises a rounded apex wall 2211 that is located at a distal end of the conical wall 2210. As exemplified, the conical wall 2210 is in the form of a truncated cone and the apex wall 2211 is rounded. In other embodiments, the conical wall 2210 may resemble a full cone that is not truncated. In such an embodiment, the apex wall 2211 may merely be considered the distal-most portion of the conical wall 2211 with no clearly visible differentiation. As discussed in greater detail below, the three-dimensional structure 2101 may be formed so that the conical wall 2210 (and the resulting cavity 2220) may have a circular transverse cross-sectional profile or an elongated/elliptical transverse cross-sectional profile.

The fingertip receiving portion 2200 further comprises a cavity 2220 having a top end that is closed by the rounded apex wall 2211. An opening 2221 into the cavity 2220 exists for receiving the tip portion of a user's finger. The opening 2221 is defined, at least in part, by a lower edge 2212 of the conical wall 2210. In the exemplified embodiment, the remainder of the opening 2221 is defined by the intersection between the fingertip receiving portion 2200 and the gripping portion 2300 because the gripping portion 2300 extends downwardly from the conical wall 2210 in an uninterrupted manner. The flap section 2301 of the gripping portion comprises a flap wall 2302 that extends from a proximal end of the conical wall 2210 of the fingertip receiving portion.

Referring now to FIG. 38, the flap wall 2311 comprises a first wall thickness t1 measured from an exposed inner surface 2312 of the flap wall 2311 to an exposed outer surface 2313 of the flap wall 2311. In the exemplified embodiment, the first wall thickness t1 varies along the length of the flap wall 2311. In another embodiment, the first wall thickness t1 is substantially constant along the length of the flap wall 2311. Similarly, the conical wall 2210 comprises a second wall thickness t2 measured from an exposed inner surface 2213 of the conical wall 2210 to an exposed outer surface 2214 of the conical wall 2210. In one embodiment, the second wall thickness t2 is less than the first wall thickness t1. As can be seen in FIG. 38, in the exemplified embodiment, the conical wall 2210 has an annular portion adjacent the rounded apex wall 2211 having the second wall thickness t2 that is less than the first wall thickness t1.

The rounded apex wall 2211 comprises a third wall thickness t3 measured from an exposed inner surface 2215 of the rounded apex wall 2211 to an exposed outer surface 2216 of the rounded apex wall 2211. In one embodiment, the third wall thickness t3 is less than the first wall thickness t1. In another embodiment, the third wall thickness t3 is greater than the second wall thickness t2. In a further embodiment, the third wall thickness t3 is greater than the second wall thickness t2 and less than the first wall thickness t1. In an even further embodiment, the third wall thickness t3 is less than the second wall thickness t2 and less than the first wall thickness t1.

As mentioned above, in certain embodiments wherein the three-dimensional structure 2101 is formed of a fibrous fabric that is press-formed, the fibers of the fibrous fabric can be subject to localized variations in stretching and/or compaction, thereby resulting in portions of the three-dimensional structure 2101 having differing stiffness. In one such embodiment, the flap wall 2301 has a first stiffness while the conical wall 2210 has a second stiffness that is greater than the first stiffness. Additionally, the rounded apex wall 2211 may have a third stiffness that is greater than the first stiffness. The third stiffness may be greater than the first stiffness and less than the second stiffness. In another embodiment, the third stiffness may be greater than the first stiffness and greater than the second stiffness.

Referring now to FIGS. 38-40 concurrently, the cone axis F-F intersects an apex of the rounded apex wall 2211. As a result, the conical wall 2210 forms a cone angle Θ1, Θ2 with the cone axis F-F. In one embodiment, the conical wall 2210 may have a circular transverse cross-sectional profile such that cone angle Θ1 and cone angle Θ2 are substantially the same. However, as in the exemplified embodiment, the conical wall 2210 has an elongated/elliptical transverse cross-sectional profile such that cone angle Θ1 is greater than cone angle Θ2. In another non-illustrated embodiment, cone angle Θ1 may be less than cone angle Θ2.

As further exemplified in the illustrated embodiment, the flap wall 2301 extends along a grip axis (not shown) that forms a grip angle Θ3 with the cone axis F-F. The grip angle Θ3 and cone angle Θ1 are the same in the exemplified embodiment. As a result, the outer surface 2303 of the flap wall 2301 forms a continuous and uninterrupted extension of the outer surface 2214 of the conical wall 2210.

The transverse cross-sectional shape and size of the cavity 2220 (and the opening 2221) will be dictated by the shape of the conical wall 2210. In one embodiment, the conical wall 2210 has an elongated/elliptical transverse cross-sectional profile such that cone angle Θ1 is greater than cone angle Θ2 as mentioned above. In such an embodiment, the cavity 2220 (and the opening 2221) will have an elliptical transverse cross-sectional profile and thus have a major axis A1 and a minor axis A2. The major axis A1 being greater than the minor axis A2. In another embodiment, the cavity 2220 (and the opening 2221) will have a circular cross-sectional profile such that A1 and A2 are substantially equal.

In the exemplified embodiment, the fingertip cleaning apparatus 2100 is a single layer structure and thus, does not include any additional structural components/layers beyond the three-dimensional structure 2101. In other embodiments, additional material layers may be added to the three-dimensional structure 2101 that is described above, and this has been discussed above in some of the earlier described embodiments.

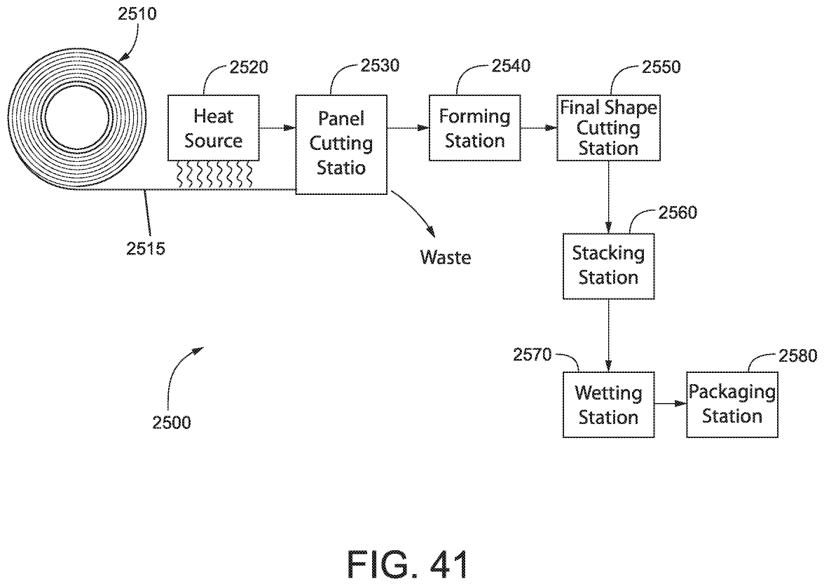

Referring now to FIG. 41, a schematic rendering of a manufacturing line 2500 for making the fingertip cleaning apparatus 2100 is shown. A roll of a sheet fabric 2510 is provided. A sheet of fabric 2515 is unrolled from the roll 2510 and is first subjected to heat that is generated by a heat source. The sheet of fabric 2515 is subjected to the heat for a period of time sufficient to raise the temperature of the sheet of fabric 2515 to a desired temperature, which may be above ambient temperature. Suitable heating sources include heated plates, hot air blowers, light sources (such as infra-red), microwaves, etc.

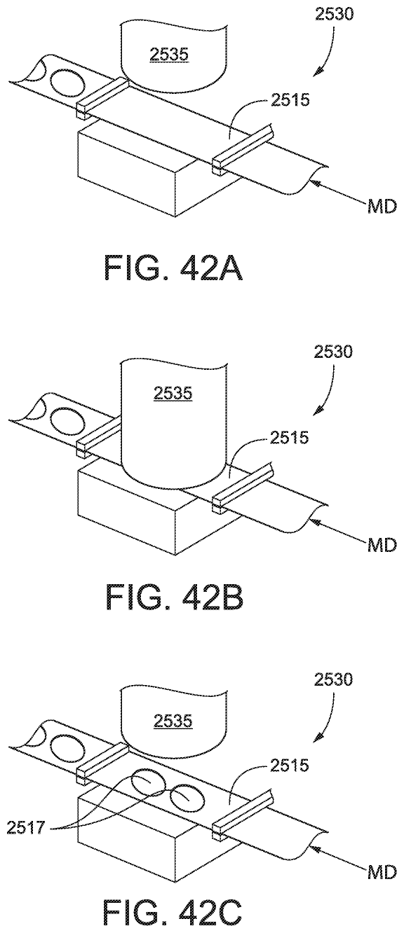

Referring now to FIGS. 41 and 42A-C concurrently, the heated sheet of fabric 2515 is then fed to a panel cutting station 2530. The feed direction is indicated by arrow MD. The panel cutting station 2530 comprises a cutting machine that may be in the form of a die cutter/stamper 2535. The flat and heated sheet of fabric 2515 is held in place under the die cutter 2535 (see FIG. 42A). The die cutter 2535 is then activated, thereby lowering so that its cutting edges/dies cut though the heated sheet of fabric 2515 (see FIG. 41B). As a result, one or more flat panels 2517 of the fabric are cut out of the sheet of fabric 2515 and released therefrom. In the exemplified embodiment, two flat panels 2517 of the fabric are cut out/created simultaneously. As shown the two flat panels 2517 are oval/elliptical in shape. However, other shapes can be selected. Additionally, other cutting tools can be used to cut the flat panels 2517 out of the sheet 2315.

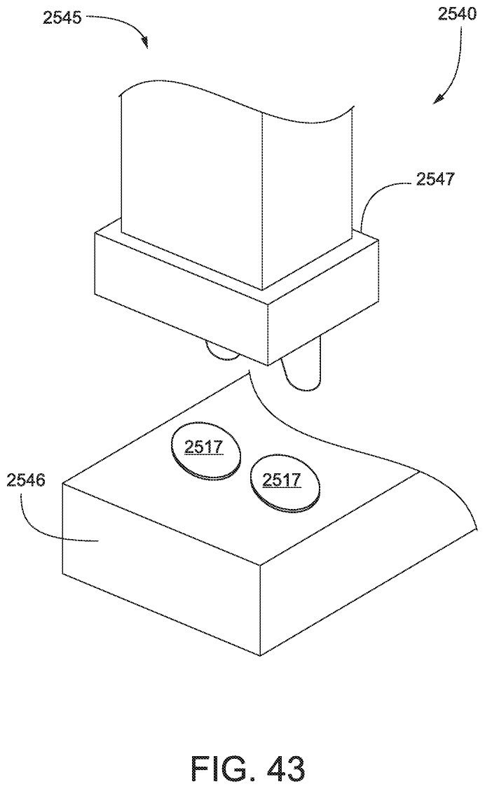

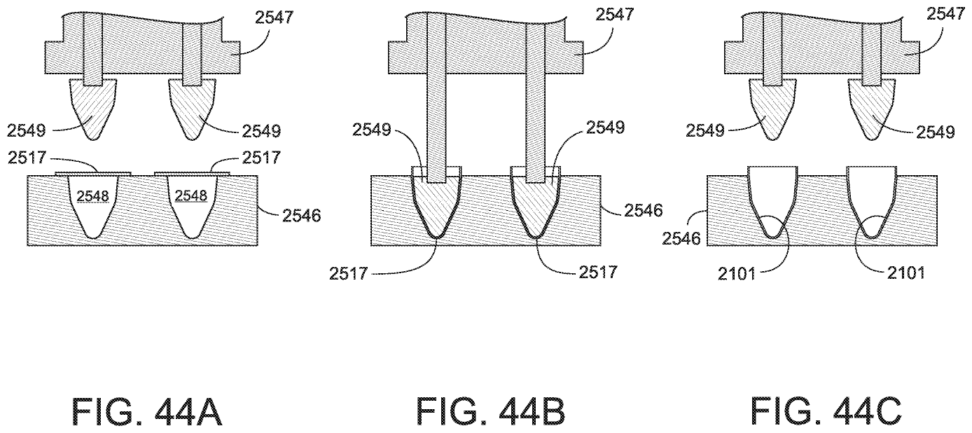

Referring now to FIGS. 41, 43 and 44A-C concurrently, the flat panels 2517 of fabric (which may have some bend/contour as result of the cutting station 2530), are then fed to a forming station 2540. The forming station 2540 includes a press 2545 that is configured to form the flat panels 2517 into the three-dimensional structures 2101. The press 2545 comprises a female mold 2546 and a corresponding male mold 2547. The female mold 2546 comprises open mold cavities 2548 that are sized and shaped to generally correspond to the desired three-dimensional structure/shape 2101. The flat panels 2517 are positioned above and overlie the open mold cavities 2548, as shown in FIG. 44A.

Once the flat panels 2517 are properly positioned over the open mold cavities 2548, the male mold 2547 is activated. As a result, male mold plungers 2549 are actuated downwardly, thereby pressing the flat panels 2517 into the open mold cavities 2548, as shown in FIG. 44B. The male mold plungers 2549 are sized and shaped to generally correspond to the size and shape of the open mold cavities 2548. Of course, the male mold plungers 2549 and the open mold cavities 2548 are designed so that a specifically designed tolerance/gap exists between the outer surfaces of the male mold plungers 2549 and the wall surfaces of the open mold cavities 2548 so that the three-dimensional structures 2101 (having the shape and properties discussed above with respect to FIGS. 33-40) are formed.

Once fully inserted into the open mold cavities 2548, the male mold plungers 2549 exert considerable compression force onto the flat panels 2517 (which have now taken the three-dimensional shape of the tolerance/gap that exists between the outer surfaces of the male mold plungers 2549 and the wall surfaces of the open mold cavities 2548). As a result of these compression forces (and possibly by the insertion of the male mold plungers 2549 and the wall surfaces of the open mold cavities 2548 at rapid speed), certain portions of the flat panels 2517 may become stretched and/or permanently compressed so that the flat panels 2517 permanently take on the shape and properties of the three-dimensional structures 2101 discussed above. The heated nature of the flat panels 2517 during the press-forming process may assist with the permanent deformation of the flat panels 2517 so that they retain the shape of three-dimensional structures 2101. Once the formation of the three-dimensional structures 2101 is complete, the male mold plungers 2549 are withdrawn from the open mold cavities 2548 (see FIG. 44C).

It should be noted that to the extent it is desired to texture all or a portion of the three-dimensional structures 2101, this can be done in the press 2545 by providing dimples or other desired texturing topography on one or both of the male mold plungers 2549 and/or the wall of the open mold cavity 2548. In one embodiment, the male mold plungers 2549 and/or the wall of the open mold cavity 2548 are configured to apply a texture to only the gripping portion 2300 of the three-dimensional structures 2101, thereby leaving the outer surface of the fingertip receiving portion smooth and substantially free of macroscopic texturing.

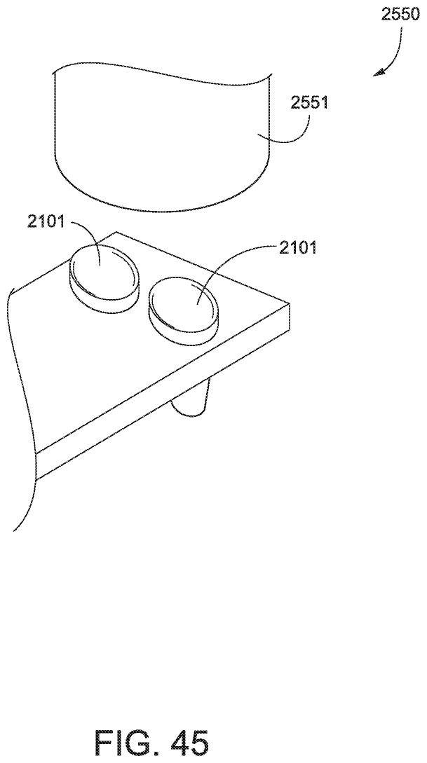

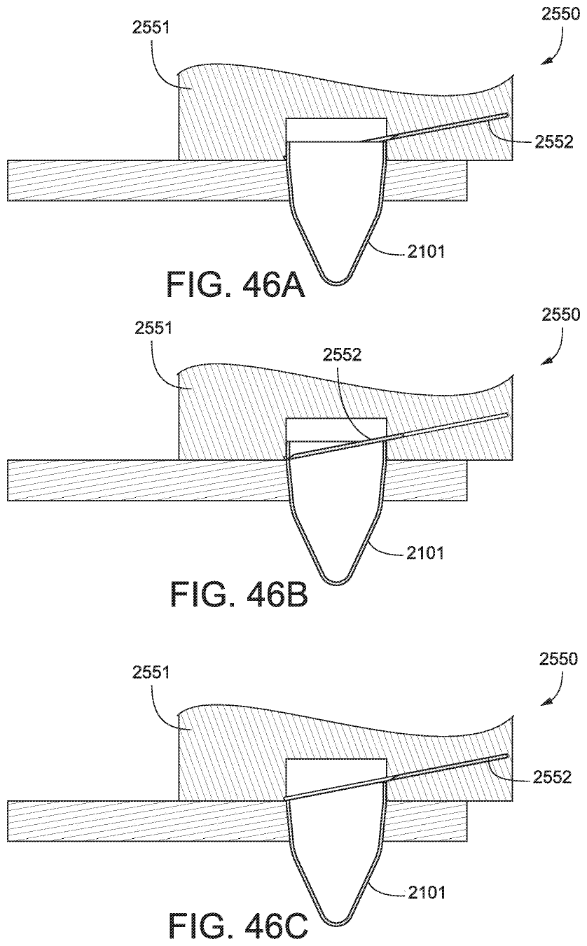

Referring now to FIGS. 41, 45 and 46A-C concurrently, the three-dimensional structures 2101 are removed from the forming station 2540 and moved to a final shape cutting station 2550. While having the desired shape-retaining conical structure, the three-dimensional structures 2101 formed in the forming station 2540 may not have the final shape/profile desired. Thus, in the final shape cutting station 2550, the three-dimensional structures 2101 are positioned and retained within a final cutting tool 2551. A blade 2552 of the final cutting tool 2551 is then used to cut the three-dimensional structures 2101 into their final shape. For example, the blades 2552 may trim a desired profile into the three-dimensional structures 2101 so that the griping portion 2300 is formed. This cutting may occur when the three-dimensional structures 2101 temporarily assume a flattened state (not shown).

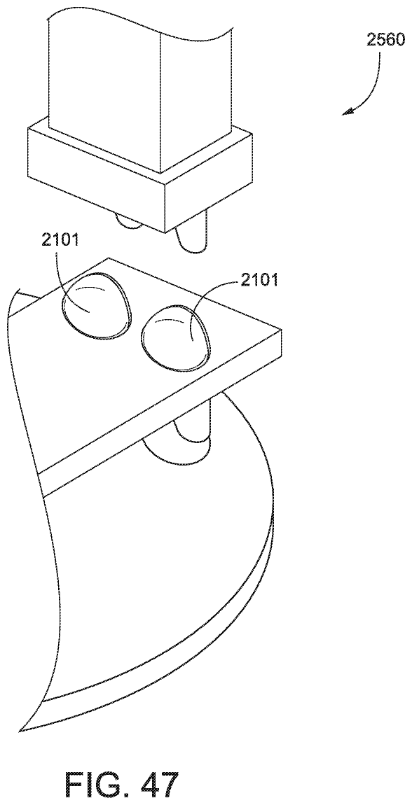

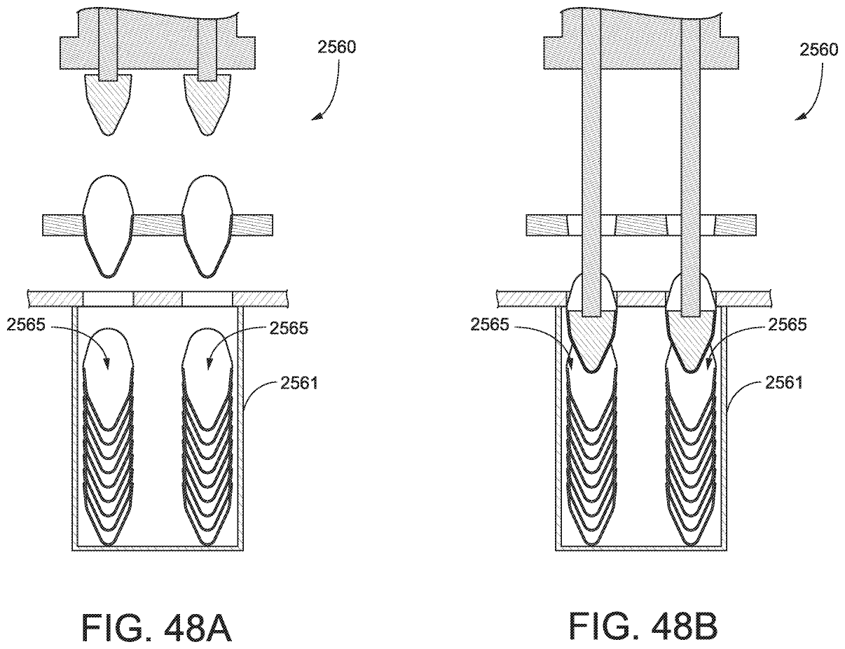

Referring now to FIGS. 47 and 48A-B concurrently, once the three-dimensional structures 2101 have been cut to their final shape, they are moved to a product stacking station 2560. At the product stacking station 2560, the three-dimensional structures 2101 are gripped and inserted into a basket 2561 in stacked and nesting arrangements 2565 as shown in FIGS. 48A-B.

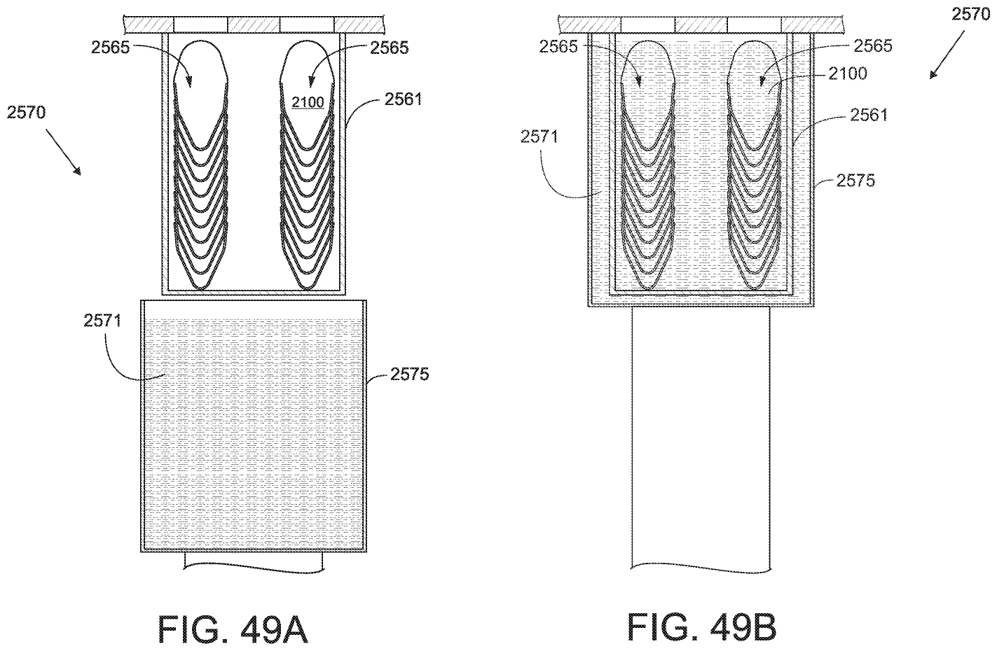

Referring now to FIGS. 49A-B, once in the stacked and nesting arrangements 2565, the products are entered into a wetting station 2570. In the wetting station 2570, the stacked and nesting arrangements 2565 are immersed in a bath 2575 of a desired liquid 2571. This can be done by either lowering the basket 2561 into the bath or by raising the bath 2575 so that the basket 2561 is immersed therein. Once in the bath of liquid 2571, the fingertip cleaning apparatus 2100 of the stacked and nesting arrangements 2565 absorb the liquid into their three-dimensional structure 2101 due to the adsorbent nature of the fibrous material of which they are constructed. In one embodiment, the liquid 2571 (which is absorbed into and forms part of the fingertip cleaning apparatus 100) is an aqueous skin cleaning composition comprising by weight, 98.687% purified water, 0.3% glycerine, 0.2% phenoxyethanol, 0.15% aloe vera, 0.15% chamomilla recutita extract, 0.1% polyaminopropyl biguanide, 0.1% polysorbate 20, 0.1% xylitol, 0.05% tocopheryl ecetate, 0.03% 2-brom-2nitropropoane-1, 3-diol, 0.03% citric acid, and 0.103% fragrance.

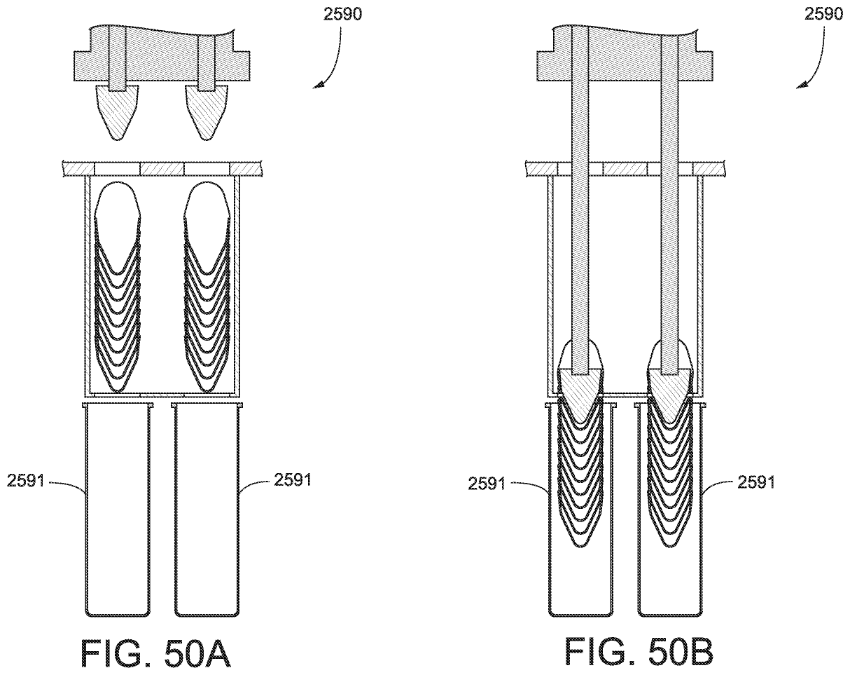

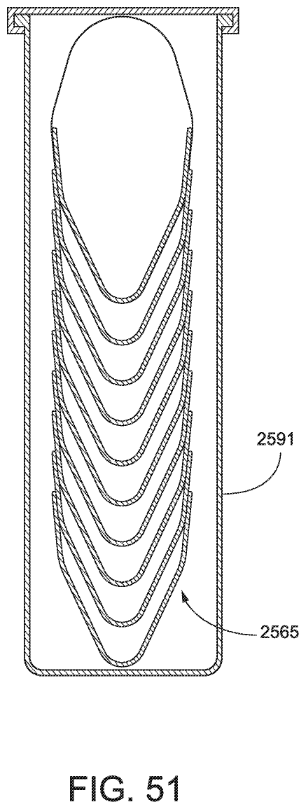

Referring now to FIGS. 50A-B and 51, once wetted the stacked and nesting arrangements 2565 are moved to a product packaging station 2590 where they are inserted into tube packages 2591 and sealed for shipping.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly.

1-51. (canceled)

52. A fingertip cleaning apparatus comprising:
a three-dimensional structure comprising:
a fingertip receiving portion comprising a conical wall, a rounded apex wall at a distal end of the conical wall, a cavity having a top end that is closed by the rounded apex wall, and an opening into the cavity defined, at least in part, by a lower edge of the conical wall; and
a gripping portion comprising a flap wall that extends from a proximal end of the conical wall of the fingertip receiving portion;
wherein the three-dimensional structure is formed from a single sheet of fabric press molded into a shape-retaining structure.

53. The fingertip cleaning apparatus according to claim 52 wherein the flap wall comprises a first wall thickness measured from an inner surface of the flap wall to an outer surface of the flap wall; and wherein at least one of the rounded apex wall and the conical wall comprises a second wall thickness measured from an inner surface of the fingertip receiving portion to an outer surface of the fingertip receiving portion, the second wall thickness being less than the first wall thickness.

54. The fingertip cleaning apparatus according to claim 53 wherein the conical wall comprises the second wall thickness.

55. The fingertip cleaning apparatus according to claim 54 wherein the rounded apex wall has a third wall thickness measured from an inner surface of the fingertip receiving portion to an outer surface of the fingertip receiving portion, the third wall thickness being greater than the second wall thickness.

56. The fingertip cleaning apparatus according to claim 54 wherein the conical wall has an annular portion adjacent the rounded apex wall having the second wall thickness.

57. The fingertip cleaning apparatus according to claim 52 wherein the flap wall has a first stiffness and the conical wall has a second stiffness that is greater than the first stiffness.

58. The fingertip cleaning apparatus according to claim 52 wherein the conical wall circumscribes a cone axis that intersects an apex of the rounded apex wall; and wherein the conical wall forms a cone angle with the cone axis and the flap wall extends along a grip axis that forms a grip angle with the cone axis, the grip angle and cone angle being substantially the same.

59. The fingertip cleaning apparatus according to claim 52 wherein in a free state, the fingertip cleaning apparatus retains the three-dimensional structure.

60. The fingertip cleaning apparatus according to claim 52 wherein the opening into the cavity is substantially oval.

61. The fingertip cleaning apparatus according to claim 60 wherein the opening into the cavity is substantially circular.

62. The fingertip cleaning apparatus according to claim 52 wherein the single panel sheet of fabric is a nonwoven fibrous sheet.

63. The fingertip cleaning apparatus according to claim 52 wherein the fingertip cleaning apparatus is free of seams.

64. The fingertip cleaning apparatus according to claim 52 wherein an outer surface of the rounded apex portion is smooth and an outer surface of the flap wall is textured.

65. The fingertip cleaning apparatus according to claim 52 wherein the single panel of fabric is an absorbent material and the three-dimensional structure is loaded with a skin cleaning liquid.

66. The fingertip cleaning apparatus according to claim 65 wherein the skin cleaning liquid is an aqueous solution comprising phenoxyethanol.

67. A fingertip cleaning apparatus comprising:
a three-dimensional structure comprising:
a fingertip receiving portion comprising a conical wall, an apex wall at a distal end of the conical wall, a cavity having a top end that is closed by the apex wall, and an opening into the cavity defined, at least in part, by a lower edge of the conical wall; and
a gripping portion comprising a flap wall that extends from a proximal end of the conical wall of the fingertip receiving portion; and
wherein the three-dimensional structure is free of seams.

68. A fingertip cleaning apparatus comprising:
a three-dimensional structure comprising:
a fingertip receiving portion comprising a conical wall, an apex wall at a distal end of the conical wall, a cavity having a top end that is closed by the apex wall, and an opening into the cavity defined, at least in part, by a lower edge of the conical wall; and
a gripping portion comprising a flap wall that extends from a proximal end of the conical wall of the fingertip receiving portion, the flap wall comprising a first wall thickness measured from an exposed inner surface of the flap wall to an exposed outer surface of the flap wall; and
wherein at least one of the apex wall and the conical wall comprise a second wall thickness measured from an exposed inner surface of the fingertip receiving portion to an exposed outer surface of the fingertip receiving portion, the second wall thickness being less than the first wall thickness.

69. (canceled)
